(19) United States
(12) Patent Application Publication
(10) Pub. No.: US 2026/0191916 A1
(43) Pub. Date: Jul. 9, 2026

(54) MICROBIAL CONSORTIA

(71) Applicant: Kanvas Biosciences, Inc., Monmouth Junction, NJ (US)

(72) Inventors: James BERLEMAN, Monmouth Junction, NJ (US); Aditya BHALLA, Monmouth Junction, NJ (US); Philip BURNHAM, Monmouth Junction, NJ (US); Matthew CHENG, Monmouth Junction, NJ (US); Peter DIEBOLD, Monmouth Junction, NJ (US); Anjali DOSHI, Monmouth Junction, NJ (US); Kyle JACOBY, Monmouth Junction, NJ (US); Hao SHI, Monmouth Junction, NJ (US); Lee SWEM, Monmouth Junction, NJ (US)

(73) Assignee: Kanvas Biosciences, Inc., Monmouth Junction, NJ (US)

(21) Appl. No.: 19/381,263

(22) Filed: Nov. 6, 2025

Related U.S. Application Data
(63) Continuation of application No. PCT/US2025/054096, filed on Nov. 5, 2025.
(60) Provisional application No. 63/716,540, filed on Nov. 5, 2024.
(60) Provisional application No. 63/740,454, filed on Dec. 31, 2024.

(51) Int. Cl. A61K 35/741; A61K 9/00; A61K 9/10; A61K 47/10; A61P 35/04; C12N 1/20; C12R 1/01

(52) U.S. Cl. CPC A61K 35/741; A61K 9/0053; A61K 9/10; A61K 47/10; A61P 35/04; C12N 1/20; C12R 2001/01

(57) ABSTRACT

The present disclosure provides microbial consortia that are useful as therapeutics, for example, for treating cancer and/or reducing tumor volume or tumor growth in patients.

### CROSS-REFERENCE TO RELATED APPLICATION

This application is a Continuation Application of International Patent Application No. PCT/US2025/054096, filed Nov. 5, 2025, which claims priority to U.S. Provisional Application No. 63/716,540, filed Nov. 5, 2024, and U.S. Provisional Application No. 63/740,454, filed Dec. 31, 2024, the contents of each of which are hereby incorporated by reference in their entirety, and to each of which priority is claimed.

### SEQUENCE LISTING

The specification further incorporates by reference the Sequence Listing submitted herewith via mail on Mar. 26, 2026. Pursuant to 37 C.F.R. §§ 1.831-1.185 and 1.839, the Sequence Listing xml file, identified as 0915920179, is 239,042,560 bytes and was created on Mar. 26, 2026. The Sequence Listing does not extend beyond the scope of the specification and thus does not contain new matter.

### BACKGROUND

Studies have demonstrated that the composition of the microbiome can impact patient health across various conditions, including the existence of, or vulnerability to, infectious diseases, autoimmune disorders, and gastrointestinal issues and conditions such as inflammatory bowel disease. Furthermore, introducing favorable microbiomes to patients can produce positive effects for a variety of therapeutic and prophylactic purposes. The present disclosure provides microbial consortia that are useful as therapeutics, for example, for treating cancer and/or reducing tumor volume or tumor growth in patients.

### SUMMARY

The present disclosure provides microbial consortia useful as therapeutics for treating cancer and/or reducing tumor volume or tumor growth in patients.

In certain non-limiting embodiments, the present disclosure provides a microbial consortium comprising or consisting of:
(a) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica; or
(b) FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908.

In certain non-limiting embodiments, the present disclosure provides a microbial consortium comprising or consisting of:
(a) Ruminococcus FBI01839, Parabacteroides distasonis, Blautia caecimuris, Longibaculum muris, Streptococcus salivarius, Blautia wexlerae FBI01866, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Enterococcus avium, Streptococcus mutans, Bacteroides uniformis FBI02111, Phocaeicola dorei, Streptococcus parasanguinis, Streptococcus FBI02176, Streptococcus parasanguinis, Enterococcus gallinarum, Streptococcus gallolyticus, Thomasclavelia ramose, Bacteroides thetaiotaomicron, Lactonifactor FBI02339, UBA1417 FBI02365, Alistipes onderdonkii, Veillonella atypica FBI02412, Extibacter hylemonae, Odoribacter FBI02593, and Bacteroides stercoris; or
(b) FBI01839, FBI01840, FBI01848, FBI01859, FBI01863, FBI01866, FBI01876, FBI01883, FBI01900, FBI01907, FBI01925, FBI01930, FBI01952, FBI01956, FBI01957, FBI02069, FBI02074, FBI02083, FBI02101, FBI02106, FBI02111, FBI02144, FBI02158, FBI02176, FBI02208, FBI02271, FBI02288, FBI02305, FBI02325, FBI02339, FBI02365, FBI02408, FBI02412, FBI02555, FBI02593, and FBI02695.

In certain non-limiting embodiments, the present disclosure provides a microbial consortium comprising or consisting of:
(a) Ruminococcus FBI01839, Parabacteroides distasonis, Blautia caecimuris, Longibaculum muris, Streptococcus salivarius, Blautia wexlerae FBI01866, Blautia massiliensis, Alistipes putredinis, Akkermansia muciniphila, Faecalimonas umbilicate, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Enterococcus avium, Streptococcus mutans, Bacteroides uniformis FBI02111, Phocaeicola dorei, Streptococcus parasanguinis, Streptococcus FBI02176, Streptococcus parasanguinis, Enterococcus gallinarum, Streptococcus gallolyticus, Thomasclavelia ramosa, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, and Bacteroides stercoris; or
(b) FBI01839, FBI01840, FBI01848, FBI01859, FBI01863, FBI01866, FBI01876, FBI01883, FBI01900, FBI01907, FBI01925, FBI01930, FBI01952, FBI01956, FBI01957, FBI02069, FBI02074, FBI02083, FBI02101, FBI02106, FBI02111, FBI02144, FBI02158, FBI02176, FBI02208, FBI02271, FBI02288, FBI02305, FBI02325, FBI02339, FBI02365, FBI02408, FBI02412, FBI02417, FBI02538, FBI02555, FBI02593, and FBI02695.

In certain non-limiting embodiments, the present disclosure provides a microbial consortium comprising or consisting of:
(a) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Bacteroides uniformis FBI02111, Phocaeicola dorei, Thomasclavelia ramosa, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, and Bacteroides stercoris; or
(b) FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, and FBI02695.

In certain non-limiting embodiments, the present disclosure provides a microbial consortium comprising or consisting of:
(a) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Bacteroides uniformis FBI02111, Phocaeicola dorei, Thomasclavelia ramosa, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, Bacteroides stercoris, and Faecalibacterium prausnitzii; or
(b) FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, and FBI02902.

In certain non-limiting embodiments, the present disclosure provides a microbial consortium comprising or consisting of:
(a) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Bacteroides uniformis FBI02111, Phocaeicola dorei, Thomasclavelia ramosa, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, Bacteroides stercoris, Blautia wexlerae FBI01861, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, Veillonella atypica FBI02908, and Enterococcus avium; or
(b) FBI01839, FBI01840, FBI01848, FBI01859, FBI01861, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, FBI02908, and FBI02101.

In certain non-limiting embodiments, the present disclosure provides a microbial consortium comprising or consisting of the species listed in Consortia 5.1 in Table 3.

In certain non-limiting embodiments, the present disclosure provides a microbial consortium comprising or consisting of the bacterial strains listed in Consortia 5.1 in Table 3.

In certain non-limiting embodiments, the present disclosure provides a microbial consortium comprising or consisting of:
(a) Ruminococcus FBI01839, Parabacteroides distasonis, Blautia caecimuris, Longibaculum muris, Blautia wexlerae, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica; or
(b) FBI01839, FBI01840, FBI01848, FBI01859, FBI01861, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908.

In certain non-limiting embodiments, the present disclosure provides a microbial consortium comprising or consisting of the species listed in Consortia 6.1 in Table 3.

In certain non-limiting embodiments, the present disclosure provides a microbial consortium comprising or consisting of the bacterial strains listed in Consortia 6.1 in Table 3.

In certain non-limiting embodiments, the present disclosure provides a microbial consortium comprising or consisting of the species listed in Consortia 6.2 in Table 3.

In certain non-limiting embodiments, the present disclosure provides a microbial consortium comprising or consisting of the bacterial strains listed in Consortia 6.2 in Table 3.

In certain non-limiting embodiments, the present disclosure provides a microbial consortium comprising or consisting of:
(a) at least 39 bacterial strains selected from the group consisting of FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908;
(b) at least 29 bacterial strains selected from the group consisting of FBI01839, FBI01840, FBI01848, FBI01859, FBI01863, FBI01866, FBI01876, FBI01883, FBI01900, FBI01907, FBI01925, FBI01930, FBI01952, FBI01956, FBI01957, FBI02069, FBI02074, FBI02083, FBI02101, FBI02106, FBI02111, FBI02144, FBI02158, FBI02176, FBI02208, FBI02271, FBI02288, FBI02305, FBI02325, FBI02339, FBI02365, FBI02408, FBI02412, FBI02555, FBI02593, and FBI02695;
(c) at least 30 bacterial strains selected from the group consisting of FBI01839, FBI01840, FBI01848, FBI01859, FBI01863, FBI01866, FBI01876, FBI01883, FBI01900, FBI01907, FBI01925, FBI01930, FBI01952, FBI01956, FBI01957, FBI02069, FBI02074, FBI02083, FBI02101, FBI02106, FBI02111, FBI02144, FBI02158, FBI02176, FBI02208, FBI02271, FBI02288, FBI02305, FBI02325, FBI02339, FBI02365, FBI02408, FBI02412, FBI02417, FBI02538, FBI02555, FBI02593, and FBI02695;
(d) at least 23 bacterial strains selected from the group consisting of FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, and FBI02695;
(e) at least 24 bacterial strains selected from the group consisting of FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, and FBI02902;
(f) at least 40 bacterial strains selected from the group consisting of FBI01839, FBI01840, FBI01848, FBI01859, FBI01861, FBI01876, FBI01883, FBI01900, FBI01907, FBI01925, FBI01930, FBI01952, FBI01956, FBI01957, FBI02069, FBI02074, FBI02083, FBI02101, FBI02111, FBI02144, FBI02305, FBI02325, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908;
(g) at least 40 bacterial strains selected from the group consisting of FBI01839, FBI01840, FBI01848, FBI01859, FBI01861, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908;
(h) at least 39 bacterial strains selected from the bacterial strains listed in Consortia 6.2 in Table 3;
(i) at least 40 bacterial strains selected from the bacterial strains listed in Consortia 6.1 in Table 3; or
(j) at least 40 bacterial strains selected from the bacterial strains listed in Consortia 5.1 in Table 3.

In certain non-limiting embodiments, the present disclosure provides a microbial consortium comprising or consisting of bacterial strains set forth in Table 3.

In certain non-limiting embodiments, the present disclosure provides a microbial consortium comprising or consisting of bacterial strains set forth in Consortia 1 of Table 3.

In certain non-limiting embodiments, the present disclosure provides a microbial consortium comprising or consisting of bacterial strains set forth in Consortia 2 of Table 3.

In certain non-limiting embodiments, the present disclosure provides a microbial consortium comprising or consisting of bacterial strains set forth in Consortia 3 of Table 3.

In certain non-limiting embodiments, the present disclosure provides a microbial consortium comprising or consisting of bacterial strains set forth in Consortia 4 of Table 3.

In certain non-limiting embodiments, the present disclosure provides a microbial consortium comprising or consisting of bacterial strains set forth in Consortia 5 of Table 3.

In certain non-limiting embodiments, the present disclosure provides a microbial consortium comprising or consisting of bacterial strains set forth in Consortia 6 of Table 3.

In certain non-limiting embodiments, the present disclosure provides a microbial consortium comprising or consisting of bacterial strains set forth in Consortia 7 of Table 3.

In certain non-limiting embodiments, the present disclosure provides a microbial consortium comprising or consisting of the microbial species set forth in Consortia 1 of Table 3.

In certain non-limiting embodiments, the present disclosure provides a microbial consortium comprising or consisting of the microbial species set forth in Consortia 2 of Table 3.

In certain non-limiting embodiments, the present disclosure provides a microbial consortium comprising or consisting of the microbial species set forth in Consortia 3 of Table 3.

In certain non-limiting embodiments, the present disclosure provides a microbial consortium comprising or consisting of the microbial species set forth in Consortia 4 of Table 3.

In certain non-limiting embodiments, the present disclosure provides a microbial consortium comprising or consisting of the microbial species set forth in Consortia 5 of Table 3.

In certain non-limiting embodiments, the present disclosure provides a microbial consortium comprising or consisting of the microbial species set forth in Consortia 6 of Table 3.

In certain non-limiting embodiments, the present disclosure provides a microbial consortium comprising or consisting of the microbial species set forth in Consortia 7 of Table 3.

In certain non-limiting embodiments, the present disclosure provides a microbial consortium comprising or consisting of bacterial strains having identical functions or substantially identical functions to the strains of the microbial consortium disclosed herein.

In certain non-limiting embodiments, the present disclosure provides a microbial consortium comprising or consisting of bacterial strains having identical functions or substantially identical functions to the bacterial strains set forth in Consortia 1 of Table 3.

In certain non-limiting embodiments, the present disclosure provides a microbial consortium comprising or consisting of bacterial strains having identical functions or substantially identical functions to the bacterial strains set forth in Consortia 2 of Table 3.

In certain non-limiting embodiments, the present disclosure provides a microbial consortium comprising or consisting of bacterial strains having identical functions or substantially identical functions to the bacterial strains set forth in Consortia 3 of Table 3.

In certain non-limiting embodiments, the present disclosure provides a microbial consortium comprising or consisting of bacterial strains having identical functions or substantially identical functions to the bacterial strains set forth in Consortia 4 of Table 3.

In certain non-limiting embodiments, the present disclosure provides a microbial consortium comprising or consisting of bacterial strains having identical functions or substantially identical functions to the bacterial strains set forth in Consortia 5 of Table 3.

In certain non-limiting embodiments, the present disclosure provides a microbial consortium comprising or consisting of bacterial strains having identical functions or substantially identical functions to the bacterial strains set forth in Consortia 6 of Table 3.

In certain non-limiting embodiments, the present disclosure provides a microbial consortium comprising or consisting of bacterial strains having identical functions or substantially identical functions to the bacterial strains set forth in Consortia 7 of Table 3.

In certain non-limiting embodiments, the present disclosure provides a microbial consortium comprising or consisting of bacterial strains having identical functions or substantially identical functions to the microbial species set forth in Consortia 1 of Table 3.

In certain non-limiting embodiments, the present disclosure provides a microbial consortium comprising or consisting of bacterial strains having identical functions or substantially identical functions to the microbial species set forth in Consortia 2 of Table 3.

In certain non-limiting embodiments, the present disclosure provides a microbial consortium comprising or consisting of bacterial strains having identical functions or substantially identical functions to the microbial species set forth in Consortia 3 of Table 3.

In certain non-limiting embodiments, the present disclosure provides a microbial consortium comprising or consisting of bacterial strains having identical functions or substantially identical functions to the microbial species set forth in Consortia 4 of Table 3.

In certain non-limiting embodiments, the present disclosure provides a microbial consortium comprising or consisting of bacterial strains having identical functions or substantially identical functions to the microbial species set forth in Consortia 5 of Table 3.

In certain non-limiting embodiments, the present disclosure provides a microbial consortium comprising or consisting of bacterial strains having identical functions or substantially identical functions to the microbial species set forth in Consortia 6 of Table 3.

In certain non-limiting embodiments, the present disclosure provides a microbial consortium comprising or consisting of bacterial strains having identical functions or substantially identical functions to the microbial species set forth in Consortia 7 of Table 3.

In certain non-limiting embodiments, the present disclosure provides a microbial consortium comprising or consisting of bacterial strains functionally equivalent or metabolically equivalent to the strains of the microbial consortium disclosed herein.

In certain non-limiting embodiments, the present disclosure provides a microbial consortium comprising or consisting of bacterial strains functionally equivalent or metabolically equivalent to the bacterial strains set forth in Consortia 1 of Table 3.

In certain non-limiting embodiments, the present disclosure provides a microbial consortium comprising or consisting of bacterial strains functionally equivalent or metabolically equivalent to the bacterial strains set forth in Consortia 2 of Table 3.

In certain non-limiting embodiments, the present disclosure provides a microbial consortium comprising or consisting of bacterial strains functionally equivalent or metabolically equivalent to the bacterial strains set forth in Consortia 3 of Table 3.

In certain non-limiting embodiments, the present disclosure provides a microbial consortium comprising or consisting of bacterial strains functionally equivalent or metabolically equivalent to the bacterial strains set forth in Consortia 4 of Table 3.

In certain non-limiting embodiments, the present disclosure provides a microbial consortium comprising or consisting of bacterial strains functionally equivalent or metabolically equivalent to the bacterial strains set forth in Consortia 5 of Table 3.

In certain non-limiting embodiments, the present disclosure provides a microbial consortium comprising or consisting of bacterial strains functionally equivalent or metabolically equivalent to the bacterial strains set forth in Consortia 6 of Table 3.

In certain non-limiting embodiments, the present disclosure provides a microbial consortium comprising or consisting of bacterial strains functionally equivalent or metabolically equivalent to the bacterial strains set forth in Consortia 7 of Table 3.

In certain non-limiting embodiments, the present disclosure provides a microbial consortium comprising or consisting of bacterial strains functionally equivalent or metabolically equivalent to the microbial species set forth in Consortia 1 of Table 3.

In certain non-limiting embodiments, the present disclosure provides a microbial consortium comprising or consisting of bacterial strains functionally equivalent or metabolically equivalent to the microbial species set forth in Consortia 2 of Table 3.

In certain non-limiting embodiments, the present disclosure provides a microbial consortium comprising or consisting of bacterial strains functionally equivalent or metabolically equivalent to the microbial species set forth in Consortia 3 of Table 3.

In certain non-limiting embodiments, the present disclosure provides a microbial consortium comprising or consisting of bacterial strains functionally equivalent or metabolically equivalent to the microbial species set forth in Consortia 4 of Table 3.

In certain non-limiting embodiments, the present disclosure provides a microbial consortium comprising or consisting of bacterial strains functionally equivalent or metabolically equivalent to the microbial species set forth in Consortia 5 of Table 3.

In certain non-limiting embodiments, the present disclosure provides a microbial consortium comprising or consisting of bacterial strains functionally equivalent or metabolically equivalent to the microbial species set forth in Consortia 6 of Table 3.

In certain non-limiting embodiments, the present disclosure provides a microbial consortium comprising or consisting of bacterial strains functionally equivalent or metabolically equivalent to the microbial species set forth in Consortia 7 of Table 3.

In certain non-limiting embodiments, the present disclosure provides a microbial consortium comprising or consisting of bacterial strains producing the same metabolites or substantially the same metabolites as the strains of the microbial consortium disclosed herein.

In certain non-limiting embodiments, the present disclosure provides a microbial consortium comprising or consisting of bacterial strains producing the same metabolites or substantially the same metabolites as the bacterial strains set forth in Consortia 1 of Table 3.

In certain non-limiting embodiments, the present disclosure provides a microbial consortium comprising or consisting of bacterial strains producing the same metabolites or substantially the same metabolites as the bacterial strains set forth in Consortia 2 of Table 3.

In certain non-limiting embodiments, the present disclosure provides a microbial consortium comprising or consisting of bacterial strains producing the same metabolites or substantially the same metabolites as the bacterial strains set forth in Consortia 3 of Table 3.

In certain non-limiting embodiments, the present disclosure provides a microbial consortium comprising or consisting of bacterial strains producing the same metabolites or substantially the same metabolites as the bacterial strains set forth in Consortia 4 of Table 3.

In certain non-limiting embodiments, the present disclosure provides a microbial consortium comprising or consisting of bacterial strains producing the same metabolites or substantially the same metabolites as the bacterial strains set forth in Consortia 5 of Table 3.

In certain non-limiting embodiments, the present disclosure provides a microbial consortium comprising or consisting of bacterial strains producing the same metabolites or substantially the same metabolites as the bacterial strains set forth in Consortia 6 of Table 3.

In certain non-limiting embodiments, the present disclosure provides a microbial consortium comprising or consisting of bacterial strains producing the same metabolites or substantially the same metabolites as the bacterial strains set forth in Consortia 7 of Table 3.

In certain non-limiting embodiments, the present disclosure provides a microbial consortium comprising or consisting of bacterial strains producing the same metabolites or substantially the same metabolites as the microbial species set forth in Consortia 1 of Table 3.

In certain non-limiting embodiments, the present disclosure provides a microbial consortium comprising or consisting of bacterial strains producing the same metabolites or substantially the same metabolites as the microbial species set forth in Consortia 2 of Table 3.

In certain non-limiting embodiments, the present disclosure provides a microbial consortium comprising or consisting of bacterial strains producing the same metabolites or substantially the same metabolites as the microbial species set forth in Consortia 3 of Table 3.

In certain non-limiting embodiments, the present disclosure provides a microbial consortium comprising or consisting of bacterial strains producing the same metabolites or substantially the same metabolites as the microbial species set forth in Consortia 4 of Table 3.

In certain non-limiting embodiments, the present disclosure provides a microbial consortium comprising or consisting of bacterial strains producing the same metabolites or substantially the same metabolites as the microbial species set forth in Consortia 5 of Table 3.

In certain non-limiting embodiments, the present disclosure provides a microbial consortium comprising or consisting of bacterial strains producing the same metabolites or substantially the same metabolites as the microbial species set forth in Consortia 6 of Table 3.

In certain non-limiting embodiments, the present disclosure provides a microbial consortium comprising or consisting of bacterial strains producing the same metabolites or substantially the same metabolites as the microbial species set forth in Consortia 7 of Table 3.

In certain embodiments, each strain of a consortia described herein comprises a 16s RNA that is at least 80% identical to a nucleotide sequence selected from SEQ ID NOs: 1-720. In certain embodiments, each strain of Consortia 1, 2, 3, 4, 5, 6, or 7 as set forth in Table 3 comprises a 16s RNA that is at least 80% identical to a nucleotide sequence selected from SEQ ID NOs: 1-720.

In certain non-limiting embodiments, each strain of a consortia described herein comprises a 16s RNA selected from SEQ ID NOs: 1-720. In certain non-limiting embodiments, each strain of Consortia 1, 2, 3, 4, 5, 6, or 7 as set forth in Table 3 comprises a 16s RNA selected from SEQ ID NOs: 1-720. In certain non-limiting embodiments, each strain of Consortia 1, 2, 3, 4, 5, 6, or 7 as set forth in Table 3 comprises a 16s RNA of the corresponding bacterial strain listed in Table 1. In certain non-limiting embodiments, each strain of Consortia 1, 2, 3, 4, 5, 6, or 7 as set forth in Table 3 comprises a 16s RNA of the corresponding species listed in Table 1.

In certain non-limiting embodiments, the present disclosure provides a pharmaceutical composition comprising the microbial consortium disclosed herein. In certain embodiments, the composition comprises up to about 10¹³ viable cells. In certain embodiments, the composition comprises a total amount of microbial consortium of up to 500 mg of total dry weight.

In certain non-limiting embodiments, the present disclosure provides a food product comprising the microbial consortium disclosed herein. In certain embodiments, the food product comprises up to about 10¹³ viable cells. In certain embodiments, the food product comprises a total amount of microbial consortium of up to 500 mg of total dry weight. In certain embodiments, the food product further comprises a prebiotic.

In certain non-limiting embodiments, the present disclosure provides a method of treating a cancer in a subject in need thereof, the method comprising administering an effective amount of the microbial consortium disclosed herein, the composition thereof, or the food product thereof.

In certain embodiments, the microbial consortium comprises or consists of:
(a) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica; or
(b) FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908;
(c) bacterial strains producing the same metabolites or substantially the same metabolites as the strains listed in (b);
(d) bacterial strains that are functionally equivalent or metabolically equivalent to the strains listed in (b);
(e) bacterial strains having identical functions or substantially identical functions to the strains listed in (b);
(f) bacterial species producing the same metabolites or substantially the same metabolites as the species listed in (a);
(g) bacterial species that are functionally equivalent or metabolically equivalent to the species listed in (a); or
(h) bacterial species having identical functions or substantially identical functions to the species listed in (a).

In certain non-limiting embodiments, the present disclosure provides a method of treating a cancer in a subject in need thereof, the method comprising administering an effective amount of the microbial consortium disclosed herein, the composition thereof, or the food product thereof.

In certain embodiments, the microbial consortium comprises or consists of:
(a) the bacterial strains listed for Consortia 1, 2, 3, 4, 5, or 6 as set forth in Table 3; or
(b) The bacterial species listed for Consortia 1, 2, 3, 4, 5, or 6 as set forth in Table 3.

In certain non-limiting embodiments, the present disclosure provides a method of reducing tumor size in a subject in need thereof, the method comprising administering an effective amount of the microbial consortium disclosed herein, the composition thereof, or the food product thereof.

In certain embodiments, the microbial consortium comprises or consists of:
(a) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica;
(b) FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908;
(c) bacterial strains producing the same metabolites or substantially the same metabolites as the strains listed in (b);
(d) bacterial strains that are functionally equivalent or metabolically equivalent to the strains listed in (b);
(e) bacterial strains having identical functions or substantially identical functions to the strains listed in (b);
(f) bacterial species producing the same metabolites or substantially the same metabolites as the species listed in (a);
(g) bacterial species that are functionally equivalent or metabolically equivalent to the species listed in (a); or
(h) bacterial species having identical functions or substantially identical functions to the species listed in (a).

In certain non-limiting embodiments, the present disclosure provides a method of reducing tumor size in a subject in need thereof, the method comprising administering an effective amount of the microbial consortium disclosed herein, the composition thereof, or the food product thereof.

In certain embodiments, the microbial consortium comprises or consists of:
(a) the bacterial strains listed for Consortia 1, 2, 3, 4, 5, or 6 as set forth in Table 3; or
(b) The bacterial species listed for Consortia 1, 2, 3, 4, 5, or 6 as set forth in Table 3.

In certain non-limiting embodiments, the present disclosure provides a method of slowing tumor growth in a subject in need thereof, the method comprising administering an effective amount of the microbial consortium disclosed herein, the composition thereof, or the food product thereof. In certain embodiments, the microbial consortium comprises or consists of:
(a) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica;
(b) FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908;
(c) bacterial strains producing the same metabolites or substantially the same metabolites as the strains listed in (b);
(d) bacterial strains that are functionally equivalent or metabolically equivalent to the strains listed in (b);
(e) bacterial strains having identical functions or substantially identical functions to the strains listed in (b);
(f) bacterial species producing the same metabolites or substantially the same metabolites as the species listed in (a);
(g) bacterial species that are functionally equivalent or metabolically equivalent to the species listed in (a); or
(h) bacterial species having identical functions or substantially identical functions to the species listed in (a).

In certain non-limiting embodiments, the present disclosure provides a method of slowing tumor growth in a subject in need thereof, the method comprising administering an effective amount of the microbial consortium disclosed herein, the composition thereof, or the food product thereof. In certain embodiments, the microbial consortium comprises or consists of:
(a) the bacterial strains listed for Consortia 1, 2, 3, 4, 5, or 6 as set forth in Table 3; or
(b) The bacterial species listed for Consortia 1, 2, 3, 4, 5, or 6 as set forth in Table 3.

In certain embodiments, the subject has received an immunotherapy. In certain embodiments, the subject has a tumor or cancer relapse. In certain embodiments, the subject is a non-responder to immunotherapy.

In certain embodiments, the immunotherapy comprises immune checkpoint inhibitors (ICI), tumor infiltrating lymphocyte (TIL) therapy, or CAR-T therapy. In certain embodiments, the immunotherapy comprises an immune checkpoint inhibitor (ICI). In certain embodiments, the ICI comprises an anti-PD1 antibody, an anti-PD-L1 antibody, an anti-CTLA-4 antibody, an anti-BTLA antibody, an anti-TIM3 antibody, an anti-LAG-3 antibody, or a combination thereof. In certain embodiments, the ICI comprises an anti-PD1 antibody and an anti-CTLA-4 antibody. In certain embodiments, the anti-PD1 antibody comprises pembrolizumab, nivolumab, cemiplimab, dostarlimab, retifanlimab-dlwr, tislelizumab, or combinations thereof. In certain embodiments, the anti-CTLA4 antibody comprises ipilimumab or tremelimumab, or a combination thereof. In certain embodiments, the anti-PD-L1 antibody comprises atezolizumab, avelumab, durvalumab, or a combination thereof.

In certain embodiments, the method comprises administering a loading dose comprising the microbial consortium, pharmaceutical composition thereof, or food product thereof, and one or more maintenance doses comprising the microbial consortium, pharmaceutical composition thereof, or food product thereof. In certain embodiments, the method further comprises administering at least one antibacterial agent, antiviral agent, antifungal agent, anti-inflammatory agent, immunosuppressive agent, prebiotic, or a combination thereof. In certain embodiments, the microbial consortium, pharmaceutical composition thereof, or food product thereof is administered orally or rectally. In certain embodiments, the microbial consortium or pharmaceutical composition thereof is administered via colonoscope.

In certain embodiments, the tumor or cancer is selected from prostate cancer, liver cancer, renal cancer, lung cancer, breast cancer, colorectal cancer, pancreatic cancer, brain cancer, hepatocellular carcinoma, lymphoma, leukemia, gastric cancer, cervical cancer, ovarian cancer, thyroid cancer, melanoma, head and neck cancer, skin cancer, and soft tissue sarcoma. In certain embodiments, the tumor or cancer is metastatic or malignant. In certain embodiments, the tumor or cancer is selected from melanoma, mismatch repair-deficient/microsatellite instability-high (dMMR/MSI-H) colorectal cancer, non-small cell lung cancer, renal cell carcinoma, head and neck squamous cell carcinoma, urothelial carcinoma, cervical cancer, and triple-negative breast cancer.

In certain non-limiting embodiments, the present disclosure provides a kit comprising the microbial consortium disclosed herein, the composition thereof, or the food product thereof. In certain embodiments, the kit comprises a container comprising a desiccant. In certain embodiments, the container comprises anaerobic conditions. In certain embodiments, the container is a blister. In certain embodiments, the kit further comprises written instructions for administering the composition or microbial consortium.

In certain non-limiting embodiments, the present disclosure provides a method of manufacturing a microbial consortium comprising: (a) obtaining a first bacterial co-culture (DS1), a second bacterial co-culture (DS2), and a third bacterial co-culture (DS3); and (b) co-culturing DS1, DS2, and DS3 in conditions sufficient for the growth of bacterial strain and/or bacterial species. In certain embodiments, the microbial consortium comprises or consists of: (i) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica; or (ii) FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908.

In certain embodiments, DS1 comprises fast growing strains, DS2 comprises medium growing strains, and DS3 comprises slow growing strains. In certain embodiments, each of DS1, DS2, and DS3 comprises bacterial strain and/or species listed in FIG. 27. In certain embodiments, the DS3 comprises a modified YCFAC medium comprising soytone in place of casitone. In certain embodiments, the modified YCFAC medium comprises threonine and N-acetylgalactosamine.

In certain non-limiting embodiments, the present disclosure provides a method of manufacturing a microbial consortium comprising a co-culture method described herein, wherein the microbial consortium comprises or consists of: (a) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica; or (b) FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI2144, FBI02305, FBI02339, FBI02365, FBI02408, FBI02412, FBI02417, FBI2538, FBI2555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908.

In certain embodiments, the method further comprises coculturing in three (3) coculture drug substance (DS) buckets. In certain embodiments, the DS buckets are the buckets listed in FIG. 27. In certain embodiments, the method of manufacturing each DS bucket is the method described in each of FIGS. 29, 30, and 31.

In certain non-limiting embodiments, the present disclosure provides a method of growing Akkermansia muciniphila in a co-culture described herein.

### BRIEF DESCRIPTION OF THE DRAWINGS

FIG. 1 is a diagram showing the manufacturing process overview from microbes in master cell banks, to fermentation, drug substance blending and drug product formulation in capsule form or a bag of liquid concentrate.

FIG. 2 shows growth curve diagrams over time and with 7 serial dilutions (E-1 through E-7) for super donor strains FBI01839 (FIG. 2A), FBI01840 (FIG. 2B), FBI01848 (FIG. 2C), FBI01859 (FIG. 2D), FBI01861 (FIG. 2E) and FBI01863 (FIG. 2F).

FIG. 3 shows growth curve diagrams over time and with 7 serial dilutions (E-1 through E-7) for super donor strains FBI01866 (FIG. 3A), FBI01876 (FIG. 3B), FBI01883 (FIG. 3C), FBI01900 (FIG. 3D), FBI01907 (FIG. 3E) and FBI01925 (FIG. 3F).

FIG. 4 shows growth curve diagrams over time and with 7 serial dilutions (E-1 through E-7) for super donor strains FBI01930 (FIG. 4A), FBI01952 (FIG. 4B), FBI01956 (FIG. 4C), FBI01957 (FIG. 4D), FBI02069 (FIG. 4E) and FBI02074 (FIG. 4F).

FIG. 5 shows growth curve diagrams over time and with 7 serial dilutions (E-1 through E-7) for super donor strains FBI02083 (FIG. 5A), FBI02101 (FIG. 5B), FBI02106 (FIG. 5C), FBI2111 (FIG. 5D), FBI02144 (FIG. 5E) and FBI2158 (FIG. 5F).

FIG. 6 shows growth curve diagrams over time and with 7 serial dilutions (E-1 through E-7) for super donor strains FBI02176 (FIG. 6A), FBI02208 (FIG. 6B), FBI2271 (FIG. 6C), FBI02288 (FIG. 6D), FBI02305 (FIG. 6E) and FBI02325 (FIG. 6F).

FIG. 7 shows growth curve diagrams over time and with 7 serial dilutions (E-1 through E-7) for super donor strains FBI02339 (FIG. 7A), FBI02365 (FIG. 7B), FBI02408 (FIG. 7C), FBI02412 (FIG. 7D), FBI02417 (FIG. 7E) and FBI02538 (FIG. 7F).

FIG. 8 shows growth curve diagrams over time and with 7 serial dilutions (E-1 through E-7) for super donor strains FBI02555 (FIG. 8A), FBI02593 (FIG. 8B), FBI02695 (FIG. 8C), FBI02882 (FIG. 8D), FBI02884 (FIG. 8E) and FBI02887 (FIG. 8F).

FIG. 9 shows growth curve diagrams over time and with 7 serial dilutions (E-1 through E-7) for super donor strains FBI02888 (FIG. 9A), FBI02890 (FIG. 9B), FBI02891 (FIG. 9C), FBI02893 (FIG. 9D), FBI02894 (FIG. 9E) and FBI02895 (FIG. 9F).

FIG. 10 shows growth curve diagrams over time and with 7 serial dilutions (E-1 through E-7) for super donor strains FBI02896 (FIG. 10A), FBI02897 (FIG. 10B), FBI02898 (FIG. 10C), FBI02899 (FIG. 10D), FBI02900 (FIG. 10E) and FBI2901 (FIG. 10F).

FIG. 11 shows growth curve diagrams over time and with 7 serial dilutions (E-1 through E-7) for super donor strains FBI02902 (FIG. 11A), FBI02903 (FIG. 11B), FBI02904 (FIG. 11C), FBI02905 (FIG. 11D), FBI02906 (FIG. 11E) and FBI02908 (FIG. 11F).

FIG. 12 is a graph showing the effect of mouse pretreatment with fecal suspension from a super donor (circles) as compared to a benchmark ICI non-responder (gray triangles) or a benchmark ICI responder (black inverted triangles) on tumor volume in an ICI treated mouse melanoma model.

FIG. 13 is a graph comparing the effect of mouse pretreatment with fecal suspension from the super donor (circles), Microbial Consortium 1 (gray squares), Microbial Consortium 2 (black squares) and an ICI non-responder (triangles) on tumor volume in an ICI treated mouse melanoma model.

FIG. 14 is a graph comparing the effect of mouse pretreatment with fecal suspension from a benchmark ICI non responder (circles) with Microbial Consortium 2 (squares) and Microbial Consortium 5 (triangles) in an ICI treated mouse melanoma model.

FIG. 15 is a graph comparing the effect of mouse pretreatment with fecal suspension from the super donor (circles), Microbial Consortium 2 (squares), and an ICI treatment non-responder (triangles) in an ICI treated mouse model of colorectal cancer.

FIG. 16 is a graph comparing the effect of mouse pretreatment with fecal suspension from the super donor (circles), Microbial Consortium 2 (squares), and a benchmark ICI treatment responder (triangles) on tumor area in an ICI treated mouse fibrosarcoma cancer model.

FIG. 17 is a graph comparing the effects of mouse pretreatment with fecal suspension from a benchmark ICI non-responder (squares), Microbial Consortium 2 (open circles), Microbial Consortium 3 (solid triangles, Microbial Consortium 4 (solid circles) and Microbial Consortium 6 (open triangles) in the ICI treated mouse fibrosarcoma cancer model.

FIG. 18 is a plot showing tumor volume in mice at day 6 after mouse tumor injection in Microbial Consortium 2 and responder treatment groups.

FIG. 19 is a plot showing tumor volume in a breast adenocarcinoma mouse cancer model, wherein the mice had a pre-existing microbiome, 10 days after mouse tumor injection in groups who received super donor fecal microbiota transplant (FMT), Microbial Consortium 2 suspension, ICI responder FMT, ICI non-responder FMT, and no treatment.

FIG. 20 is a bar graph showing the relative abundance of bacteria identified from the super donor FMT present in clinical study recipients after FMT. The detection of the bacteria pre-FMT (left bars in each pairing on each side of a hash mark) was near zero. Post-FMT measures shows an increase in the abundance of the bacteria (right bars in each pairing) and, therefore, substantial engraftment of isolates from this super donor in the clinical trial recipients.

FIG. 21 is a plot of engraftment data for the mouse models and the human clinical patients following super donor FMT or, in mice, microbial consortium treatment. The upper plot shows engraftment in various mouse models (germ free BP melanoma and specific pathogen free (SPF) MC38 colorectal cancer models). Community Treated is treated with Microbial Consortium 2, except for the top Study 333 data in Germ-Free BP melanoma mice, in which the mice were treated with Microbial Consortium 1. SD Stool Treated is super donor FMT treatment. Each vertical column represents a bacterial isolate. The lower plot shows the results of human studies and each row represents a patient. The identities of the isolates in each column align with those of the mouse models and super donor. Correlate data on % Prevalence and % Mean Abundance in the super donor stool sample itself is provided at the top of the plot.

FIG. 22 is a schematic image showing phylogenetic trees. The left hand image shows the phylogenetic tree for the Super Donor Microbiome. The key phyla are shaded. The largest group in the lower right is Bacillota. Then, moving in a counter-clockwise direction, the other phyla Bacteroidota, Actinomycetota, and Verrucomicrobiota. the right hand image shows the phylogenetic tree for the Super Donor Isolates.

FIG. 23 is a schematic of the dosing schedule for mice as described in Example 9.

FIG. 24 shows a comparison of the ability of Consortia 7 to reduce tumor area compared to the control of a non-responder FMT over time, showing a significant trend of reduced tumor size post dosing.

FIG. 25 shows the tumor size comparison of treatment with Consortia 7 compared to a non-responder FMT at day 6.

FIG. 26 shows that there was no negative effect on the weight of the mice upon administration of Consortia 7.

FIG. 27 shows the final DSs that were designed for optimal growth for Consortia 7.

FIGS. 28A-28C show growth curves and bucketing of each of the strains of Consortia 7 into DS1-3.

FIG. 29 depicts the DS1 co-culture fermentation process for the 20 strains included in DS1 of Consortia 7.

FIG. 30 depicts the DS2 co-culture fermentation process for the 19 strains included in DS2 of Consortia 7.

FIG. 31 depicts the DS3 co-culture fermentation process for the 20 strains included in DS3 of Consortia 7.

FIG. 32 depicts the initial experiment to grow DS3.

FIG. 33 depicts the optimized method of growing DS3 for Consortia 7.

### DETAILED DESCRIPTION

The present disclosure provides microbial consortia useful as therapeutics for treating cancer and/or reducing tumor volume or tumor growth in patients. The microbial species present in each of the consortia were chosen based on identification of microbial species present in fecal material donations from a super donor cancer survivor. Given that the species identified were from a single donor, each microbial consortium containing some or all of these species has species that can be compatible and cooperate to form a functional ecosystem. All references cited are herein incorporated by reference.

### Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

It is understood that aspects and embodiments of the present disclosure described herein include “comprising,” “consisting,” and “consisting essentially of” aspects and embodiments. The terms “comprises” and “comprising” are intended to have the broad meaning ascribed to them in U.S. Patent Law and can mean “includes,” “including” and the like. To facilitate an understanding of the present disclosure, a number of terms and phrases are defined below.

The term “a” and “an” as used herein mean “one or more” and include the plural unless the context is appropriate.

By “fecal microbiome” of a subject is meant the community of microorganisms that exists in a fecal specimen from the subject.

By “microbial consortium,” is meant a mixture of two or more isolated species of microbes that have been cultured.

By “fecal microbiota transplant” or “FMT” is meant the transplant of a mixture of two or more bacterial species derived from the fecal material of a donor and transplanted to a recipient.

By “super donor,” is meant a subject that responds to a treatment and has a favorable property in their fecal material that converts another subject who has been a non-responder to the treatment into a responder after FMT from the super donor.

By “responder,” is meant a subject that responds to a treatment but who does not demonstrate a favorable property in their fecal material that converts another subject from a non-responder into a responder after FMT from the responder.

As used herein, the terms “gastrointestinal engraftment” or “engraft” or “engraftment” refer to the establishment of one or more than one microbe, or microbial consortia, in one or more niches of the gastrointestinal tract that, prior to administration of the microbial consortium, were not present or detectable. Engraftment may be transient or may be persistent.

As used herein, the term “supportive community” refers to one or more microbial species that, when administered with an active microbe, enhance one or more characteristics of the active microbe selected from the group consisting of gastrointestinal engraftment, biomass, metabolic substrate metabolism, and longitudinal stability.

As used herein, the term “pharmaceutical composition” refers to the combination of an active agent with a carrier, inert or active, making the composition especially suitable for therapeutic use in vivo or ex vivo.

As used herein, the term “pharmaceutically acceptable carrier” refers to any of the standard pharmaceutical carriers, such as phosphate-buffered saline solution, water, emulsions (e.g., oil/water or water/oil emulsions), and various types of wetting agents. The compositions also can include stabilizers and preservatives. For examples of carriers, stabilizers, and adjuvants, see e.g., Martin, Remington's Pharmaceutical Sciences, 15th Ed. Mack Publ. Co., Easton, Pa. [1975].

As used herein, the term “metabolism,” “metabolize,” “metabolization,” or variants thereof refers to the biochemical conversion of a metabolic substrate to a metabolic product. In certain embodiments, metabolization includes isomerization.

As used herein, the term “biomass,” refers to the total mass of one or more microbes, or consortia, in a given area or volume.

As used herein, the term “microbial consortium” refers to a mixture of two or more isolated microbial strains that are expanded in culture, wherein one microbial strain in the mixture has a beneficial or desired effect on another microbial strain in the mixture.

As used herein, the term “subject” refers to an organism to be treated by the microbial consortium and compositions described herein. Such organisms preferably include, but are not limited to, mammals (e.g., murines, simians, equines, bovines, porcines, canines, felines, and the like), and more preferably include humans.

As used herein, the terms “tumor,” “cancer,” and “neoplasia” are used interchangeably and refer to cells exhibiting uncontrolled growth, proliferation, or survival. The term “malignancy” refers to the ability of such cells to invade adjacent or surrounding tissue. The term “metastasis” refers to the spread or dissemination of a tumor, cancer, or neoplasia to one or more secondary sites, locations, or regions within the subject, wherein the secondary sites are distinct from the primary tumor or cancer.

The term percent “identity” or “sequence identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

When used in reference to 16S rRNA sequences, a “sequence identity” of at least 97% indicates that two microbial strains are likely to belong to the same species, whereas 16S rRNA sequences having less than 97% sequence identity indicate that two microbial strains likely belong to different species, and 16S rRNA sequences having less than 95% sequence identity indicates that two microbial strains likely belong to distinct genera (Stackebrandt E., and Goebel, B. M., Int J Syst Bact, 44 (1994) 846-849).

As used herein, the terms “functional equivalent” or “functionally equivalent” refers to microbes, microbial consortia, and compositions that share similar or identical role (e.g., metabolism of oxalate). For example, without any limitation, two different microbial consortium that can catalyze high concentration of oxalate are functional equivalent to each other.

As used herein, the term “probiotic” refers to live microorganisms which, when administered in adequate amounts, confer a health benefit on the host. Further information on probiotics can be found in Hill et al., Nature reviews Gastroenterology & hepatology 11.8 (2014): 506-514.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

### Microbial Consortium

The present disclosure described herein provides microbial consortia. A microbial consortium comprises a plurality of active microbes and can also have an effective amount of a supportive community of microbes.

In certain embodiments, each microbial consortium disclosed herein comprises all, or a subset, of the bacterial species listed in Table 1. The listing includes genus and species designations. For species that are novel, the bacteria are identified by genus in combination with an FBI identifier. In one exemplary embodiment, a novel genus was identified; this genus is referenced by its closest known relative, UBA1417, together with its corresponding FBI identifier. Sequencing of these organisms generated multiple genomic DNA sequences, which are represented by the respective SEQ ID NO identifiers provided herein.

In certain embodiments, each microbial consortium disclosed herein comprises all, or a subset, of the bacterial strains listed in Table 1. The listing includes genus and species designations. For species that are novel, the bacteria are identified by genus in combination with an FBI identifier.

In certain embodiments, each microbial consortium disclosed herein comprises all, or a subset, of the following bacterial strains listed in Table 1: FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908.

In certain embodiments, each microbial consortium disclosed herein comprises all, or a subset, of the following bacterial species listed in Table 1: Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica.

In certain embodiments, each microbial consortium disclosed herein does not include strain FBI02325 (Bacteroides thetaiotaomicron). In certain embodiments, each microbial consortium disclosed herein does not include Bacteroides thetaiotaomicron.

In certain embodiments, each microbial consortium disclosed herein does not include FBI01861 and/or FBI01866 (Blautia wexlerae). In certain embodiments, each microbial consortium disclosed herein does not the species Blautia wexlerae.

In certain embodiments, each microbial consortium disclosed herein does not include FBI01863 (Streptococcus salivarius). In certain embodiments, each microbial consortium disclosed herein does not include the species Streptococcus salivarius.

In certain embodiments, each microbial consortium disclosed herein does not include one or more of FBI02158 (Streptococcus parasanguinis), FBI02176 (Streptococcus FBI02176), FBI02208 (Streptococcus parasanguinis), FBI02271 (Enterococcus gallinarum), and/or FBI02288 (Streptococcus gallolyticus). In certain embodiments, each microbial consortium disclosed herein does not include one or more of the species Streptococcus parasanguinis, Streptococcus FBI02176, Streptococcus parasanguinis, Enterococcus gallinarum, and/or Streptococcus gallolyticus.

In certain embodiments, each microbial consortium disclosed herein does not include FBI02901 (Agathobacter rectalis). In certain embodiments, each microbial consortium disclosed herein does not include the species Agathobacter rectalis.

In certain embodiments, each microbial consortium disclosed herein includes FBI02897 (Phocaeicola vulgatus). In certain embodiments, each microbial consortium disclosed herein includes the species Phocaeicola vulgatus.

TABLE 1
Bacterial components of microbial consortia

| Bacteria | | |
|---|---|---|
| ID | Species | SEQ ID NOS |
| FBI01839 | Ruminococcus FBI01839 | 1 |
| FBI01840 | Parabacteroides | 2-9 |
| | distasonis | |
| FBI01848 | Blautia caecimuris | 10 |
| FBI01859 | Longibaculum muris | 11 |
| FBI01861 | Blautia wexlerae | 12 |
| FBI01863 | Streptococcus salivarius | 13 |
| FBI01866 | Blautia wexlerae | 14 |
| FBI01876 | Blautia massiliensis | 15 |
| FBI01883 | Alistipes putredinis | 16 |
| FBI01900 | Akkermansia | 17-35 |
| | muciniphila | |
| FBI01907 | Faecalimonas umbilicata | 36-38 |
| FBI01925 | Clostridium scindens | 39 |
| FBI01930 | Bacteroides | 40-43 |
| | cellulosilyticus | |
| FBI01952 | Eggerthella lenta | 44-74 |
| FBI01956 | Monoglobus | 75 |
| | pectinilyticus | |
| FBI01957 | Gordonibacter | 76-83 |
| | urolithinfaciens | |
| FBI02069 | Bacteroides ovatus | 84-88 |
| FBI02074 | Bacteroides | 89-92 |
| | thetaiotaomicron | |
| FBI02083 | Bacteroides uniformis | 93-150 |
| FBI02101 | Enterococcus avium | 151-152 |
| FBI02106 | Streptococcus mutans | 153 |
| FBI02111 | Bacteroides uniformis | 154-158 |
| FBI02144 | Phocaeicola dorei | 159-206 |
| FBI02158 | Streptococcus | 207-208 |
| | parasanguinis | |
| FBI02176 | Streptococcus FBI02176 | 209-211 |
| FBI02208 | Streptococcus | 212-213 |
| | parasanguinis | |
| FBI02271 | Enterococcus gallinarum | 214 |
| FBI02288 | Streptococcus | 215-218 |
| | gallolyticus | |
| FBI02305 | Thomasclavelia ramosa | 219-247 |
| FBI02325 | Bacteroides | 248-272 |
| | thetaiotaomicron | |
| FBI02339 | Lactonifactor FBI02339 | 273-277 |
| FBI02365 | UBA1417 FBI02365 | 278-310 |
| FBI02408 | Alistipes onderdonkii | 311-333 |
| FBI02412 | Veillonella atypica | 334-335 |
| FBI02417 | Raoultibacter | 336 |
| | massiliensis | |
| FBI02538 | Flavonifractor plautii | 337-425 |
| FBI02555 | Extibacter hylemonae | 426-456 |
| FBI02593 | Odoribacter FBI02593 | 457 |
| FBI02695 | Bacteroides stercoris | 458-487 |
| FBI02882 | Enterocloster | 488-540 |
| | clostridioformis | |
| FBI02884 | Hungatella hathewayii | 541-542 |
| FBI02887 | Scatavimonas FBI02887 | 543-549 |
| FBI02888 | Ruminococcus gnavus | 550-574 |
| FBI02890 | Enterocloster bolteae | 575-581 |
| FBI02891 | Blautia FBI02891 | 582-583 |
| FBI02893 | Bacteroides ovatus | 584-595 |
| FBI02894 | Bacteroides finegoldii | 596-606 |
| FBI02895 | Luoshenia tenuis | 607 |
| FBI02896 | Bacteroides caccae | 608-617 |
| FBI02897 | Phocaeicola vulgatus | 618-635 |
| FBI02898 | Parabacteroides merdae | 636-641 |
| FBI02899 | Lacticaseibacillus | 642-643 |
| | paracasei | |
| FBI02900 | Oliverpabstia intestinalis | 644-654 |
| FBI02902 | Faecalibacterium | 667-702 |
| | prausnitzii | |
| FBI02903 | Bifidobacterium longum | 703-705 |
| FBI02904 | Bifidobacterium bifidum | 706 |
| FBI02905 | Lawsonibacter | 707-710 |
| | asaccharolyticus | |
| FBI02906 | Bifidobacterium dentium | 711 |
| FBI02908 | Veillonella atypica | 712-720 |
| FBI02901 | Agathobacter rectalis | 655-666 |

In certain embodiments, subsets of species can include those able to produce short chain fatty acids and/or modify bile acids, utilize specific types of nutrients, occupy certain niches, cross-feed, degrade mucin, protect against disease, and/or provide therapeutic benefit.

In certain embodiments, the microbial consortium comprises at least 50%, 55%, 60%, 65%, 70%, 75%, 80%, 85% 90%, or 95% of the bacterial species selected from Ruminococcus FBI01839, Parabacteroides distasonis, Blautia caecimuris, Longibaculum muris, Blautia wexlerae FBI01861, Blautia wexlerae FBI01866, Blautia massiliensis, Alistipes putredinis, Akkermansia muciniphila, Faecalimonas umbilicate, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Bacteroides uniformis FBI02111, Phocaeicola dorei, Thomasclavelia ramose, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, and Bacteroides stercoris.

In certain embodiments, the microbial consortium comprises at least 50%, 55%, 60%, 65%, 70%, 75%, 80%, 85% 90%, or 95% of the bacterial species selected from Ruminococcus FBI01839, Parabacteroides distasonis, Blautia caecimuris, Longibaculum muris, Blautia wexlerae FBI01861, Blautia wexlerae FBI01866, Blautia massiliensis, Alistipes putredinis, Akkermansia muciniphila, Faecalimonas umbilicate, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Bacteroides uniformis FBI02111, Phocaeicola dorei, Thomasclavelia ramose, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica FBI-2908.

In certain embodiments, the microbial consortium comprises at least 50%, 55%, 60%, 65%, 70%, 75%, 80%, 85% 90%, or 95% of the bacterial species selected from Consortia 7 as set forth in Table 3.

In certain embodiments, the microbial consortium comprises at least 50%, 55%, 60%, 65%, 70%, 75%, 80%, 85% 90%, or 95% of the bacterial species selected from Consortia 6 as set forth in Table 3. In certain embodiments, the microbial consortium comprises at least 50%, 55%, 60%, 65%, 70%, 75%, 80%, 85% 90%, or 95% of the bacterial species selected from Consortia 5 as set forth in Table 3. In certain embodiments, the microbial consortium comprises at least 50%, 55%, 60%, 65%, 70%, 75%, 80%, 85% 90%, or 95% of the bacterial species selected from Consortia 4 as set forth in Table 3. In certain embodiments, the microbial consortium comprises at least 50%, 55%, 60%, 65%, 70%, 75%, 80%, 85% 90%, or 95% of the bacterial species selected from Consortia 3 as set forth in Table 3. In certain embodiments, the microbial consortium comprises at least 50%, 55%, 60%, 65%, 70%, 75%, 80%, 85% 90%, or 95% of the bacterial species selected from Consortia 2 as set forth in Table 3. In certain embodiments, the microbial consortium comprises at least 50%, 55%, 60%, 65%, 70%, 75%, 80%, 85% 90%, or 95% of the bacterial species selected from Consortia 1 as set forth in Table 3.

In certain embodiments, the microbial consortium does not comprise Streptococcus or Enterococcus bacteria. In certain embodiments, these bacteria can have limited engraftment in humans and in mice. In certain embodiments, these microbes are not true gut commensals (e.g., oral microbes) and/or have pathobiont characteristics.

In certain embodiments, the microbial consortium also comprises one or more of Streptococcus salivarius, Streptococcus mutans, Streptococcus parasanguinis FBI02158, Streptococcus FBI02176, Streptococcus parasanguinis FBI02208, Streptococcus gallolyticus, Enterococcus avium, and Enterococcus gallinarum.

In certain embodiments, the microbial consortium comprises Ruminococcus FBI01839, Parabacteroides distasonis, Blautia caecimuris, Longibaculum muris, Blautia wexlerae FBI01861, Blautia massiliensis, Alistipes putredinis, Akkermansia muciniphila, Faecalimonas umbilicate, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron FBI02074, Bacteroides uniformis FBI02083, Bacteroides uniformis FBI02111, Phocaeicola dorei, Thomasclavelia ramose, Bacteroides thetaiotaomicron FBI02325, Lactonifactor FBI02339, UBA1417 FBI02365, Alistipes onderdonkii, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Bacteroides ovatus, Bacteroides finegoldii, Bacteroides caccae, Phocaeicola vulgatus, Parabacteroides merdae, Oliverpabstia intestinalis, Bifidobacterium longum, Bifidobacterium bifidum, Lawsonibacter asaccharolyticus, and Agathobacter rectalis.

In certain embodiments, the microbial consortium comprises Ruminococcus FBI01839, Parabacteroides distasonis, Blautia caecimuris, Longibaculum muris, Blautia wexlerae FBI01861, Blautia massiliensis, Alistipes putredinis, Akkermansia muciniphila, Faecalimonas umbilicate, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron FBI02074, Bacteroides uniformis FBI02083, Enterococcus avium, Bacteroides uniformis FBI02111, Phocaeicola dorei, Enterococcus gallinarum, Streptococcus gallolyticus, Thomasclavelia ramose, Bacteroides thetaiotaomicron FBI02325, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Bifidobacterium longum, and Lawsonibacter asaccharolyticus.

In certain embodiments, the microbial consortium comprises Ruminococcus spp., Parabacteroides distasonis, Akkermansia muciniphila, Faecalibacterium prausnitzii, Bacteroides thetaiotaomicron, Bifidobacterium longum, Enterococcus faecium, Bacteroides fragilis, Bifidobacterium bifidum, Alistipes putredinis, Clostridium scindens, Bacteroides uniformis, Bacteroides ovatus, Agathobacter rectalis, Phocaeicola vulgatus, Enterococcus gallinarum, Blautia spp, and/or Odoribacter spp.

In certain embodiments, the microbial consortium comprises a bacterial species selected from each of the phyla Bacillota, Bacteriodota, Actinomycetota, and Verrucomicrobiota, as described in Table 1 above.

In certain embodiments, the microbial consortium comprises a microbial consortium designated as “Microbial Consortium 1” (Consortia 1 as set forth in Table 3), “Microbial Consortium 2” (Consortia 2 as set forth in Table 3), “Microbial Consortium 3 (Consortia 3 as set forth in Table 3), “Microbial Consortium 4” (Consortia 4 as set forth in Table 3), “Microbial Consortium 5” (Consortia 5 as set forth in Table 3), “Microbial Consortium 6” (Consortia 6 as set forth in Table 3), or “Microbial Consortium 7” (Consortia 7 as set forth in Table 3).

In certain embodiments, the microbial consortium consists of a microbial consortium designated as “Microbial Consortium 1” (Consortia 1 as set forth in Table 3), “Microbial Consortium 2” (Consortia 2 as set forth in Table 3), “Microbial Consortium 3 (Consortia 3 as set forth in Table 3), “Microbial Consortium 4” (Consortia 4 as set forth in Table 3), “Microbial Consortium 5” (Consortia 5 as set forth in Table 3), “Microbial Consortium 6” (Consortia 6 as set forth in Table 3), or “Microbial Consortium 7” (Consortia 7 as set forth in Table 3).

In certain embodiments, the consortia comprises or consists of Ruminococcus FBI01839, Parabacteroides distasonis, Blautia caecimuris, Longibaculum muris, Streptococcus salivarius, Blautia wexlerae FBI01866, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Enterococcus avium, Streptococcus mutans, Bacteroides uniformis FBI02111, Phocaeicola dorei, Streptococcus parasanguinis, Streptococcus FBI02176, Streptococcus parasanguinis, Enterococcus gallinarum, Streptococcus gallolyticus, Thomasclavelia ramose, Bacteroides thetaiotaomicron, Lactonifactor FBI02339, UBA1417 FBI02365, Alistipes onderdonkii, Veillonella atypica FBI02412, Extibacter hylemonae, Odoribacter FBI02593, and Bacteroides stercoris.

In certain embodiments, the consortia comprises or consists of FBI01839, FBI01840, FBI01848, FBI01859, FBI01863, FBI01866, FBI01876, FBI01883, FBI01900, FBI01907, FBI01925, FBI01930, FBI01952, FBI01956, and FBI01957, followed by FBI02069, FBI02074, FBI02083, FBI02101, FBI02106, FBI02111, FBI02144, FBI02158, FBI02176, FBI02208, FBI02271, FBI02288, FBI02305, FBI02325, FBI02339, FBI02365, FBI02408, FBI02412, FBI02555, FBI02593, and FBI02695. In certain embodiments, each microbe of this consortia comprises a 16s RNA comprising a nucleotide sequence identified in Table 1.

In certain embodiments, the consortia comprises or consists of Ruminococcus FBI01839, Parabacteroides distasonis, Blautia caecimuris, Longibaculum muris, Streptococcus salivarius, Blautia wexlerae FBI01866, Blautia massiliensis, Alistipes putredinis, Akkermansia muciniphila, Faecalimonas umbilicate, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Enterococcus avium, Streptococcus mutans, Bacteroides uniformis FBI02111, Phocaeicola dorei, Streptococcus parasanguinis, Streptococcus FBI02176, Streptococcus parasanguinis, Enterococcus gallinarum, Streptococcus gallolyticus, Thomasclavelia ramosa, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, and Bacteroides stercoris.

In certain embodiments, the consortia or consists of FBI01839, FBI01840, FBI01848, FBI01859, FBI01863, FBI01866, FBI01876, FBI01883, FBI01900, FBI01907, FBI01925, FBI01930, FBI01952, FBI01956, FBI01957, FBI02069, FBI02074, FBI02083, FBI02101, FBI02106, FBI02111, FBI02144, FBI02158, FBI02176, FBI02208, FBI02271, FBI02288, FBI02305, FBI02325, FBI02339, FBI02365, FBI02408, FBI02412, FBI02417, FBI02538, FBI02555, FBI02593, and FBI02695. In certain embodiments, each microbe of this comprises a 16s RNA comprising a nucleotide sequence identified in Table 1.

In certain embodiments, the consortia comprises or consists of Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Bacteroides uniformis FBI02111, Phocaeicola dorei, Thomasclavelia ramosa, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, and Bacteroides stercoris.

In certain embodiments, the consortia comprises or consists of BI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, and FBI02695. In certain embodiments, each microbe of this consortia comprises a 16s RNA comprising a nucleotide sequence identified in Table 1.

In certain embodiments, the consortia comprises or consists of Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Bacteroides uniformis FBI02111, Phocaeicola dorei, Thomasclavelia ramosa, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, Bacteroides stercoris, and Faecalibacterium prausnitzii.

In certain embodiments, the consortia comprises or consists of BI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, and FBI02902. In certain embodiments, each microbe of this consortia comprises a 16s RNA comprising a nucleotide sequence identified in Table 1.

In certain embodiments, the consortia comprises or consists of Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Bacteroides uniformis FBI02111, Phocaeicola dorei, Thomasclavelia ramosa, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, Bacteroides stercoris, Blautia wexlerae FBI01861, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica FBI02908.

In certain embodiments, the consortia comprises or consists of FBI01839, FBI01840, FBI01848, FBI01859, FBI01861, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908. In certain embodiments, each microbe of this consortia comprises a 16s RNA comprising a nucleotide sequence identified in Table 1.

In certain embodiments, the consortia comprises or consists of Ruminococcus FBI01839, Parabacteroides distasonis, Blautia caecimuris, Longibaculum muris, Blautia wexlerae, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Enterococcus avium, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica.

In certain embodiments, the consortia comprises or consists of FBI01839, FBI01840, FBI01848, FBI01859, FBI01861, FBI01876, FBI01883, FBI01900, FBI01907, FBI01925, FBI01930, FBI01952, FBI01956, FBI01957, FBI02069, FBI02074, FBI02083, FBI02101, FBI02111, FBI02144, FBI02305, FBI02325, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908. In certain embodiments, each microbe of this consortia comprises a 16s RNA comprising a nucleotide sequence identified in Table 1.

In certain embodiments, the consortia comprises or consists of Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica.

In certain embodiments, the consortia comprises or consists of FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908. In certain embodiments, each microbe of this consortia comprises a 16s RNA comprising a nucleotide sequence identified in Table 1.

The present disclosure provides microbial consortia 7 of Table 3. The relevance of the microbial consortia 7 of Table 3 with respect to therapeutic effect is such that the composition may include those microbial species of consortia 7 of Table 3 as the only microbial species in the composition. Consequently, the present disclosure provides microbial consortia comprising or consisting of Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica. Alternatively, the present disclosure provides microbial consortia comprising or consisting of FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908.

In certain embodiments, the microbial consortia of the present disclosure provide engraftment in recipients and can provide health and therapeutic benefits under certain conditions. In certain embodiments, the microbial consortia of the present disclosure are comprised of bacterial with survival and growth properties that are similar to those exemplified in the Examples.

In certain embodiments, a microbial consortium comprises 2 to 500 microbial species. For example, in certain non-limiting embodiments, a microbial consortium comprises 3 to 500, 4 to 500, 5 to 500, 6 to 500, 7 to 500, 8 to 500, 9 to 500, 10 to 500, 11 to 500, 12 to 500, 13 to 500, 14 to 500, 15 to 500, 16 to 500, 17 to 500, 18 to 500, 19 to 500, 20 to 500, 21 to 500, 22 to 500, 23 to 500, 24 to 500, 25 to 500, 30 to 500, 35 to 500, 40 to 500, 45 to 500, 50 to 500, 60 to 500, 70 to 500, 80 to 500, 90 to 500, 100 to 500, 110 to 500, 120 to 500, 130 to 500, 140 to 500, 150 to 500, 160 to 500, 170 to 500, 180 to 500, 190 to 500, 200 to 500, 210 to 500, 220 to 500, 230 to 500, 240 to 500, 250 to 500, 260 to 500, 270 to 500, 280 to 500, 290 to 500, 300 to 500, 400 to 500, 3 to 300, 4 to 300, 5 to 300, 6 to 300, 7 to 300, 8 to 300, 9 to 300, 10 to 300, 11 to 300, 12 to 300, 13 to 300, 14 to 300, 15 to 300, 16 to 300, 17 to 300, 18 to 300, 19 to 300, 20 to 300, 21 to 300, 22 to 300, 23 to 300, 24 to 300, 25 to 300, 30 to 300, 35 to 300, 40 to 300, 45 to 300, 50 to 300, 60 to 300, 70 to 300, 80 to 300, 90 to 300, 100 to 300, 110 to 300, 120 to 300, 130 to 300, 140 to 300, 150 to 300, 160 to 300, 170 to 300, 180 to 300, 190 to 300, 200 to 300, 210 to 300, 220 to 300, 230 to 300, 240 to 300, 250 to 300, 260 to 300, 270 to 300, 280 to 300, 290 to 300, 3 to 250, 4 to 250, 5 to 250, 6 to 250, 7 to 250, 8 to 250, 9 to 250, 10 to 250, 11 to 250, 12 to 250, 13 to 250, 14 to 250, 15 to 250, 16 to 250, 17 to 250, 18 to 250, 19 to 250, 20 to 250, 21 to 250, 22 to 250, 23 to 250, 24 to 250, 25 to 250, 30 to 250, 35 to 250, 40 to 250, 45 to 250, 50 to 250, 60 to 250, 70 to 250, 80 to 250, 90 to 250, 100 to 250, 110 to 250, 120 to 250, 130 to 250, 140 to 250, 150 to 250, 160 to 250, 170 to 250, 180 to 250, 190 to 250, 200 to 250, 210 to 250, 220 to 250, 230 to 250, 240 to 250, 3 to 200, 4 to 200, 5 to 200, 6 to 200, 7 to 200, 8 to 200, 9 to 200, 10 to 200, 11 to 200, 12 to 200, 13 to 200, 14 to 200, 15 to 200, 16 to 200, 17 to 200, 18 to 200, 19 to 200, 20 to 200, 21 to 200, 22 to 200, 23 to 200, 24 to 200, 25 to 200, 30 to 200, 35 to 200, 40 to 200, 45 to 200, 50 to 200, 60 to 200, 70 to 200, 80 to 200, 90 to 200, 100 to 200, 110 to 200, 120 to 200, 130 to 200, 140 to 200, 150 to 200, 160 to 200, 170 to 200, 180 to 200, 190 to 200, 3 to 150, 4 to 150, 5 to 150, 6 to 150, 7 to 150, 8 to 150, 9 to 150, 10 to 150, 11 to 150, 12 to 150, 13 to 150, 14 to 150, 15 to 150, 16 to 150, 17 to 150, 18 to 150, 19 to 150, 20 to 150, 21 to 150, 22 to 150, 23 to 150, 24 to 150, 25 to 150, 30 to 150, 35 to 150, 40 to 150, 45 to 150, 50 to 150, 60 to 150, 70 to 150, 80 to 150, 90 to 150, 100 to 150, 110 to 150, 120 to 150, 130 to 150, 140 to 150, 3 to 100, 4 to 100, 5 to 100, 6 to 100, 7 to 100, 8 to 100, 9 to 100, 10 to 100, 11 to 100, 12 to 100, 13 to 100, 14 to 100, 15 to 100, 16 to 100, 17 to 100, 18 to 100, 19 to 100, 20 to 100, 21 to 100, 22 to 100, 23 to 100, 24 to 100, 25 to 100, 30 to 100, 35 to 100, 40 to 100, 45 to 100, 50 to 100, 60 to 100, 70 to 100, 80 to 100, 90 to 100, 3 to 75, 4 to 75, 5 to 75, 6 to 75, 7 to 75, 8 to 75, 9 to 75, 10 to 75, 11 to 75, 12 to 75, 13 to 75, 14 to 75, 15 to 75, 16 to 75, 17 to 75, 18 to 75, 19 to 75, 20 to 75, 21 to 75, 22 to 75, 23 to 75, 24 to 75, 25 to 75, 30 to 75, 35 to 75, 40 to 75, 45 to 75, 50 to 75, 60 to 75, 70 to 75, 3 to 50, 4 to 50, 5 to 50, 6 to 50, 7 to 50, 8 to 50, 9 to 50, 10 to 50, 11 to 50, 12 to 50, 13 to 50, 14 to 50, 15 to 50, 16 to 50, 17 to 50, 18 to 50, 19 to 50, 20 to 50, 21 to 50, 22 to 50, 23 to 50, 24 to 50, 25 to 50, 30 to 50, 35 to 50, 40 to 50, 45 to 50, 3 to 25, 4 to 25, 5 to 25, 6 to 25, 7 to 25, 8 to 25, 9 to 25, 10 to 25, 11 to 25, 12 to 25, 13 to 25, 14 to 25, 15 to 25, 16 to 25, 17 to 25, 18 to 25, 19 to 25, 20 to 25, 21 to 25, 22 to 25, 23 to 25, or 24 to 25 microbial species. For example, in certain non-limiting embodiments, a microbial consortium comprises about 20 to about 200, about 70 to about 80, about 80 to about 90, about 100 to about 110, or about 150 to about 160 microbial species.

In certain embodiments, a microbial consortium described herein comprises a microbial species having a relative abundance of approximately 90%, 80%, 70%, 60%, 50%, 40%, 30%, 20%, 10%, 5%, 1%, 0.1%, 0.01%, 0.001%, 0.0001%, 0.00001%, or 0.000001% of the total microbial consortium. In certain embodiments, the relative abundance of a microbial species is determined, for example, by metagenomic sequencing, High Phylogenetic Resolution microbiome mapping by Fluorescence in situ Hybridization (HiPR-FISH), quantitative PCR (qPCR), or droplet digital PCR, and calculated as the percentage of reads that are classified as an identified microbial species, divided by the genome size or the counted number of bacterial of particular taxa divided by the total counted number of bacteria. For example, in certain non-limiting embodiments, the relative abundance of a microbial species is determined, for example, by metagenomic shotgun sequencing, HiPR-FISH, or qPCR.

### Functionally Equivalent and Identical Drug Products

The strains included in each microbial consortia disclosed herein (e.g., Consortia 1, Consortia 2, Consortia 3, Consortia 4, Consortia 5, Consortia 6, and Consortia 7, each as set forth in Table 3) are described herein by 16S RNA sequences and functional characteristics. In certain embodiments, equivalent consortia of Consortia 1-7 can be generated by screening multiple of the same strain to find equivalent strains with equivalent function to those that comprise Consortia 1-7, respectively. Accordingly, in certain embodiments identical strains may theoretically have different functions, strains can be screened using 16S RNA and Biolog to identify functionally identical and equivalent strains from any fecal collection using the methods of collection described herein.

In certain embodiments, each microbial consortium disclosed herein (e.g., Consortia 1-7, each as set forth in Table 3) was articulately designed to have multiple of the same strain in the Consortium. In certain embodiments, this provides a benefit of redundancy to ensure function. In certain embodiments, redundancy is not required for equivalent function so long as one of the otherwise redundant strains is included in the final drug product at a sufficient viable cell count amount to achieve in vivo function in a subject. Accordingly, a Consortium that is equivalent or identical to a microbial consortium disclosed herein (e.g., Consortia 1-7, each as set forth in Table 3) may contain all redundancies or alternatively may contain no or fewer redundancies per strain so long as the included strains achieve in vivo function in a subject.

In certain embodiments, the microbial consortium comprises or consists of strains having identical functions to the strains of any of Consortia 17, each as set forth in Table 3. In certain embodiments, the microbial consortium comprises or consists of strains functionally equivalent to the strains of any of Consortia 17, each as set forth in Table 3. In certain embodiments, the microbial consortium comprises or consists of strains having substantially identical functions to the strains of any of Consortia 17, each as set forth in Table 3. In certain embodiments, the microbial consortium comprises or consists of strains producing the same metabolites as the strain of any of Consortia 17, each as set forth in Table 3. In certain embodiments, the microbial consortium comprises or consists of strains producing substantially the same metabolites as the strain of any of Consortia 17, each as set forth in Table 3.

### Methods of Treatment

The present disclosure provides methods of treatment using any of the microbial consortia disclosed herein. The methods are useful for treating cancer patients and those in need of slowing tumor growth or reducing tumor size. Such treatments can be useful as a standalone therapy, as a neo-adjuvant therapy, or in combination with immunotherapy (e.g., immune checkpoint inhibitors (ICI), T-cell therapy (e.g., tumor infiltrating lymphocyte (TIL) therapy or CAR-T therapy), monoclonal antibodies, treatment vaccines, and immune system modulators). In certain embodiment, a microbial consortium of the present disclosure is administered to a cancer patient who is being treated with an ICI, for example and without any limitation, anti-PD1 therapies (e.g., pembrolizumab, nivolumab, or cemiplimab), anti PD-L1 therapies (e.g., atezolizumab, avelumab, and durvalumab), and anti-CTLA-4 therapies (e.g., ipilimumab and tremelimumab)). Treatment with a microbial consortium can precede immunotherapy by 1-2 weeks or could be administered contemporaneously. A microbial consortium of the present disclosure could also be administered in combination with chemotherapy or radiation therapy (see Kalasbail et al., Cancers (Basel) 13(18): 4623, Sep. 15, 2021; Liu et al., Genomics Proteomics Bioinformatics 21(1): 84-96, Jul. 30, 2022; Yu et al., Cancers (Basel) 14(22): 5685, Nov. 19, 2022).

In certain embodiments, with respect to patient eligibility, individuals suitable for treatment include those who have previously failed immune checkpoint inhibitor (ICI) therapy, as well as those initiating ICI therapy for the first time. Non-limiting clinical indications include conditions in which immune system functionality is important to therapeutic response including, for example but without any limitations, cancers for which ICI therapy is established yet associated with a substantial proportion of non-responders.

In certain embodiments, the presently disclosed methods and compositions can be used for the treatment of neoplastic diseases, including but not limited to benign, malignant, metastatic, and non-metastatic neoplasms, tumors, and cancers. The presently disclosed methods and compositions can be used for the treatment of neoplastic conditions at any stage (e.g., I-V) or grade (e.g., G1-G3), including those that are progressing, worsening, stabilized, or in remission. Cancers treatable according to the present disclosure encompass a wide range of anatomical origins, including but not limited to the bladder, blood, bone, bone marrow, brain, breast, colon, esophagus, gastrointestinal tract, gum, head, kidney, liver, lung, nasopharynx, neck, ovary, prostate, skin, stomach, testis, tongue, and uterus. Histological types include, without limitation, carcinomas (e.g., squamous cell, basal cell, papillary, adenocarcinoma), sarcomas (e.g., fibrosarcoma, liposarcoma, osteosarcoma), melanomas, lymphomas, leukemias, and mixed tumors. In certain embodiments, the tumor or cancer is selected from prostate cancer, liver cancer, renal cancer, lung cancer, breast cancer, colorectal cancer, pancreatic cancer, brain cancer, hepatocellular carcinoma, lymphoma, leukemia, gastric cancer, cervical cancer, ovarian cancer, thyroid cancer, melanoma, head and neck cancer, skin cancer, and soft tissue sarcoma. In certain embodiments, the tumor or cancer is metastatic or malignant.

Additional exemplary indications include, without limitation, melanoma, mismatch repair-deficient/microsatellite instability-high (dMMR/MSI-H) colorectal cancer, non-small cell lung cancer, renal cell carcinoma, head and neck squamous cell carcinoma, urothelial carcinoma, cervical cancer, and triple-negative breast cancer.

In certain embodiments, the methods of treatment disclosed herein are useful for patients experiencing adverse effects associated with other cancer therapies, including, without limitation, gastrointestinal complications such as treatment-induced colitis. Mitigation of such side effects can enable patients to maintain the primary cancer therapy, for example, immune checkpoint inhibitor (ICI) therapy, for an extended duration, thereby potentially improving clinical outcomes.

In certain embodiments, screening for treatment eligibility can include individuals exhibiting suboptimal microbiome profiles, such as those characterized by low microbial diversity, the presence of pathogens or pathobionts, and/or microbiomes significantly divergent from a microbial consortium of the present invention. However, such characteristics are not required. In certain embodiments, patients can avoid concomitant administration of proton pump inhibitors (PPIs), corticosteroids, and/or antibiotics during treatment. In certain embodiments, individuals with a recent history of microbiome-modifying interventions, including antibiotic or PPI use, may be particularly suited for the therapeutic methods described herein.

Further embodiments include patients with disrupted microbiomes, such as those undergoing colonoscopy following bowel preparation. The treatment can also be applicable to patients requiring hormone therapy or other interventions that impact microbiome composition.

In certain embodiments, the route of administration is oral, for example, via capsule or tablet. Alternative routes of delivery can include colonoscopic administration. Additional non-limiting alternatives include rectal suppository or administration via nasogastric tube.

Pre-treatment can optionally include antibiotic administration and/or colonic lavage using polyethylene glycol (PEG) or other suitable laxatives. In certain embodiments, post-treatment protocols provide that patients avoid the use of corticosteroids, proton pump inhibitors (PPIs), and/or antibiotics. Post-treatment can further include administration of loperamide or a similar agent to reduce rapid expulsion of the microbial consortium disclosed herein.

In certain embodiments, both the product and the treatment regimen are configured to support immune checkpoint inhibitor (ICI) therapy. Post-treatment microbiome diversity may represent a critical component of therapeutic success, and the product is formulated to promote such diversity. In certain embodiments, antibiotics are not administered. In certain embodiments when antibiotics are administered, reinstating a diverse microbiome following antibiotic exposure can be beneficial.

In certain embodiments, the formulation and route of delivery are modified to support robust microbial engraftment. For example, but without any limitation, frozen formulations delivered via colonoscopy can be used based on their demonstrated enhanced engraftment. Identification of key strains associated with response to ICI therapy can be facilitated using high-resolution platforms such as HiPR-FISH. In certain embodiments, a superdonor provided three samples collected across multiple timepoints, enabling identification of strains common to these samples, which can be prioritized for inclusion in the microbial consortia.

In certain embodiments, a Microbial Consortium disclosed herein (e.g., Consortia 1-7, each as set forth in Table 3) is administered as a single dose or as multiple doses. In certain embodiments, a Microbial Consortium disclosed herein (e.g., Consortia 1-7, each as set forth in Table 3) is administered once a day for 2 days, 3 days, 4 days, 5 days, 6 days, 1 week, 2 weeks 3 weeks, 1 month, 2 months, 3 months, 4 months, 5 months, 6 months, or 1 year. In certain embodiments, a Microbial Consortium disclosed herein (e.g., Consortia 1-7, each as set forth in Table 3) is administered multiple times daily. In certain embodiments, a Microbial Consortium disclosed herein (e.g., Consortia 1-7, each as set forth in Table 3) is administered twice daily, three times daily, 4 times daily, or 5 times daily. In certain embodiments, a Microbial Consortium disclosed herein (e.g., Consortia 1-7, each as set forth in Table 3) is administered intermittently. In certain embodiments, a Microbial Consortium disclosed herein (e.g., Consortia 1-7, each as set forth in Table 3) is administered once weekly, once monthly, or when a subject is in need thereof.

In certain embodiments, Consortia 7 is administered as a single dose or as multiple doses. In certain embodiments, Consortia 7 is administered once a day for 2 days, 3 days, 4 days, 5 days, 6 days, 1 week, 2 weeks 3 weeks, 1 month, 2 months, 3 months, 4 months, 5 months, 6 months, or 1 year. In certain embodiments, Consortia 7 is administered multiple times daily. In certain embodiments, Consortia 7 is administered twice daily, three times daily, 4 times daily, or 5 times daily. In certain embodiments, Consortia 7 is administered intermittently. In certain embodiments, Consortia 7 is administered once weekly, once monthly, or when a subject is in need thereof.

In certain embodiments, a Microbial Consortium disclosed herein (e.g., Consortia 1-7, each as set forth in Table 3) is administered at a first loading dose and then followed by maintenance doses. In certain embodiments, the first loading dose is administered for 1 day, 2 days, 3 days, 4 days, 5 days, 6 days, 7 days, 8 days, 9 days, or 10 days. In certain embodiments, the loading dose is administered for 1-3 days. In certain embodiments, the loading dose is administered for 2-4 days. In certain embodiments, the loading dose is administered for 2-3 days. In certain embodiments, the loading dose is administered for 3-5 days. In certain embodiments, the loading dose is administered for 4-6 days. In certain embodiments, the loading dose is administered for 5-7 days. In certain embodiments, the loading dose is administered for 1 day. In certain embodiments, the loading dose is administered for 3 days. In certain embodiments, the loading dose is administered for 2 days.

In certain embodiments, the loading dose is administered for 1 day, 2 days, 3 days, 4 days, 5 days, 6 days, 7 days, 8 days, 9 days, or 10 days after the immunotherapy. In certain embodiments, the loading dose is administered for 1-3 days after the immunotherapy. In certain embodiments, the loading dose is administered for 2-4 days after the immunotherapy. In certain embodiments, the loading dose is administered for 2-3 days after the immunotherapy. In certain embodiments, the loading dose is administered for 3-5 days after the immunotherapy. In certain embodiments, the loading dose is administered for 4-6 days after the immunotherapy. In certain embodiments, the loading dose is administered for 5-7 days after the immunotherapy. In certain embodiments, the loading dose is administered for 1 day after the immunotherapy. In certain embodiments, the loading dose is administered for 3 days after the immunotherapy. In certain embodiments, the loading dose is administered for 2 days after the immunotherapy.

In certain embodiments, the loading dose is administered daily for the duration of the immunotherapy administered to the patient.

In certain embodiments, the loading dose is administered for 1 day, 2 days, 3 days, 4 days, 5 days, 6 days, 7 days, 8 days, 9 days, or 10 days before the immunotherapy. In certain embodiments, the loading dose is administered for 1-3 days before the immunotherapy. In certain embodiments, the loading dose is administered for 2-4 days before the immunotherapy. In certain embodiments, the loading dose is administered for 2-3 days before the immunotherapy. In certain embodiments, the loading dose is administered for 3-5 days before the immunotherapy. In certain embodiments, the loading dose is administered for 4-6 days before the immunotherapy. In certain embodiments, the loading dose is administered for 5-7 days before the immunotherapy. In certain embodiments, the loading dose is administered for 1 day before the immunotherapy. In certain embodiments, the loading dose is administered for 3 days before the immunotherapy. In certain embodiments, the loading dose is administered for 2 days before the immunotherapy.

In certain embodiments, the loading dose is administered daily before the immunotherapy is administered to the patient.

In certain embodiments, the maintenance doses are administered for 5-10 days following the last loading dose. In certain embodiments, the maintenance doses are administered for 7-12 days following the last loading dose. In certain embodiments, the maintenance doses are administered for 10-14 days following the last loading dose. In certain embodiments, the maintenance doses are administered for 14-21 days following the last loading dose. In certain embodiments, the maintenance doses are administered for 21-28 days following the last loading dose. In certain embodiments, the maintenance doses are administered for 14 days following the last loading dose. In certain embodiments, the maintenance doses are administered for 21 days following the last loading dose. In certain embodiments, the maintenance doses are administered for 28 days following the last loading dose. In certain embodiments, the maintenance doses are administered for about 8 days following the last loading dose. In certain embodiments, the maintenance doses are administered for about 7 days following the last loading dose. In certain embodiments, the maintenance doses are administered for about 6 days following the last loading dose. In certain embodiments, the maintenance doses are administered for about 9 days following the last loading dose. In certain embodiments, the maintenance doses are administered for about 10 days following the last loading dose. In certain embodiments, the maintenance doses are administered for at least 21 days following the last loading dose. In certain embodiments, the maintenance doses are administered for at least 1 month following the last loading dose. In certain embodiments, the maintenance doses are administered for at least 2 months following the last loading dose. In certain embodiments, the maintenance doses are administered for at least 4 months following the last loading dose. In certain embodiments, the maintenance doses are administered for the maintenance doses are administered for at least 6 months following the last loading dose. In certain embodiments, the maintenance doses are administered for at least 8 months following the last loading dose. In certain embodiments, the maintenance doses are administered for at least 10 months following the last loading dose. In certain embodiments, the maintenance doses are administered for at least 12 months following the last loading dose. In certain embodiments, the maintenance doses are administered for at least 18 months following the last loading dose. In certain embodiments, the maintenance doses are administered for at least 24 months following the last loading dose. In certain embodiments, the loading dose is administered for 2 days and the maintenance dose is administered for 6 days (for a total of a 8-day course of treatment). In certain embodiments, the loading dose is administered for 2 days and the maintenance dose is administered for 7 days (for a total of a 9-day course of treatment). In certain embodiments, the loading dose is administered for 2 days and the maintenance dose is administered for 8 days (for a total of a 10 day course of treatment). In certain embodiments, the loading dose is administered for 9 days and the maintenance dose is administered for 9 days (for a total of a 11 day course of treatment). In certain embodiments, the loading dose is administered for 2 days and the maintenance dose is administered for 10 days (for a total of a 12 day course of treatment).

In certain embodiments, the loading dose follows the pretreatment with antibiotics as described in the Combination Therapy section below. In certain embodiments, the loading dose follows the pretreatment with a bowel preparation as described in the Combination Therapy section below. In certain embodiments, the loading dose follows the pretreatment with antibiotics and a bowel preparation as described in the Combination Therapy section below.

In certain embodiments when used in a patient that will receive an immunotherapy including an immune checkpoint inhibitor (ICI), a Microbial Consortium disclosed herein (e.g., Consortia 1-7, each as set forth in Table 3) is administered loading dose before, after, or at the same time as the immunotherapy. In certain embodiments, loading dose is administered 1 day, 2 days, 3 days, 4 days, 5 days, 6 days, 7 days, 8 days, 9 days, or 10 days before the immunotherapy. In certain embodiments, the loading dose is administered 1 day, 2 days, 3 days, 4 days, 5 days, 6 days, 7 days, 8 days, 9 days, or 10 days after the immunotherapy. In certain embodiments, the loading dose is administered at the same time as the immunotherapy. In certain embodiments, the first loading dose is administered for 1 day, 2 days, 3 days, 4 days, 5 days, 6 days, 7 days, 8 days, 9 days, or 10 days. In certain embodiments, the loading dose is administered for 1-3 days. In certain embodiments, the loading dose is administered for 2-4 days. In certain embodiments, the loading dose is administered for 2-3 days. In certain embodiments, the loading dose is administered for 3-5 days. In certain embodiments, the loading dose is administered for 4-6 days. In certain embodiments, the loading dose is administered for 5-7 days. In certain embodiments, the loading dose is administered for 1 day. In certain embodiments, the loading dose is administered for 3 days. In certain embodiments, the loading dose is administered for 2 days. In certain embodiments, the maintenance doses are administered for 5-10 days following the last loading dose. In certain embodiments, the maintenance doses are administered for 7-12 days following the last loading dose. In certain embodiments, the maintenance doses are administered for 10-14 days following the last loading dose. In certain embodiments, the maintenance doses are administered for 14-21 days following the last loading dose. In certain embodiments, the maintenance doses are administered for 21-28 days following the last loading dose. In certain embodiments, the maintenance doses are administered for 14 days following the last loading dose. In certain embodiments, the maintenance doses are administered for 21 days following the last loading dose. In certain embodiments, the maintenance doses are administered for 28 days following the last loading dose. In certain embodiments, the maintenance doses are administered for at least 21 days following the last loading dose. In certain embodiments, the maintenance doses are administered for at least 1 month following the last loading dose. In certain embodiments, the maintenance doses are administered for at least 2 months following the last loading dose. In certain embodiments, the maintenance doses are administered for at least 4 months following the last loading dose. In certain embodiments, the maintenance doses are administered for the maintenance doses are administered for at least 6 months following the last loading dose. In certain embodiments, the maintenance doses are administered for at least 8 months following the last loading dose. In certain embodiments, the maintenance doses are administered for at least 10 months following the last loading dose. In certain embodiments, the maintenance doses are administered for at least 12 months following the last loading dose. In certain embodiments, the maintenance doses are administered for at least 18 months following the last loading dose. In certain embodiments, the maintenance doses are administered for at least 24 months following the last loading dose. In certain embodiments, the maintenance doses are administered for about 8 days following the last loading dose. In certain embodiments, the maintenance doses are administered for about 7 days following the last loading dose. In certain embodiments, the maintenance doses are administered for about 6 days following the last loading dose. In certain embodiments, the maintenance doses are administered for about 9 days following the last loading dose. In certain embodiments, the maintenance doses are administered for about 10 days following the last loading dose. In certain embodiments, the loading dose is administered for 2 days and the maintenance dose is administered for 6 days (for a total of a 8-day course of treatment). In certain embodiments, the loading dose is administered for 2 days and the maintenance dose is administered for 7 days (for a total of a 9-day course of treatment). In certain embodiments, the loading dose is administered for 2 days and the maintenance dose is administered for 8 days (for a total of a 10 day course of treatment). In certain embodiments, the loading dose is administered for 9 days and the maintenance dose is administered for 9 days (for a total of a 11 day course of treatment). In certain embodiments, the loading dose is administered for 2 days and the maintenance dose is administered for 10 days (for a total of a 12 day course of treatment). In certain embodiments, the loading dose follows the pretreatment with antibiotics as described in the Combination Therapy section below. In certain embodiments, the loading dose follows the pretreatment with a bowel preparation as described in the Combination Therapy section below. In certain embodiments, the loading dose follows the pretreatment with antibiotics and a bowel preparation as described in the Combination Therapy section below.

In certain embodiments, a Consortia is administered at a loading dose and then followed by maintenance doses. In certain embodiments, the loading dose comprises about 10¹² viable cells. In certain embodiments, the loading dose comprises one or more coated enteric capsules. In certain embodiments, the one or more coated enteric capsules comprise about 10¹¹ viable cells. In certain embodiments, the loading dose comprises 10 coated enteric capsules. In certain embodiments, the loading dose is administered for 1 day. In certain embodiments, the loading dose is administered twice a day. In certain embodiments, the maintenance dose comprises about 2×10¹¹ viable cells. In certain embodiments, each maintenance dose comprises one or more coated enteric capsules. In certain embodiments, the one or more coated enteric capsules comprise about 10¹¹ viable cells. In certain embodiments, each maintenance dose comprises 2 coated enteric capsules. In certain embodiments, the maintenance doses are administered for 2 days, 3 days, 4 days, 5 days, 6 days, 7 days, 8 days, 9 days, 10 days, 11 days, 12 days, 13 days, 2 weeks, 3 weeks, 4 weeks, 5 weeks, 6 weeks, 8 weeks, 10 weeks, or 12 weeks following the last loading dose.

In certain embodiments, each capsule contains a range of 5×10¹⁰ to 5×10¹¹ viable cells/capsule. In certain embodiments, each capsule contains a range of 5×10⁹ to 5×10¹⁰ viable cells/capsule. In certain embodiments, each capsule contains a range of 5×10¹¹ to 5×10¹² viable cells/capsule. In certain embodiments, maltodextrin is included as an excipient in the capsules.

In certain embodiments, a Microbial Consortium disclosed herein (e.g., Consortia 1-7, each as set forth in Table 3) can be administered via an enteric route. For example, in certain non-limiting embodiments, a microbial consortium is administered orally, rectally (e.g., by enema, suppository, or colonoscope), or by oral or nasal tube.

In certain embodiments, a Microbial Consortium disclosed herein (e.g., Consortia 1-7, each as set forth in Table 3) is administered orally. In certain embodiments, the oral administration is by a powder. In certain embodiments, the oral administration is by a slurry. In certain embodiments, the oral administration is by pills or capsules.

In certain embodiments, a Microbial Consortium disclosed herein (e.g., Consortia 1-7, each as set forth in Table 3) can be administered to a specific location along the gastrointestinal tract. For example, in certain non-limiting embodiments, a microbial consortium can be administered into one or more gastrointestinal location including the mouth, esophagus, stomach, small intestine (duodenum, jejunum, ileum), large intestine (cecum, ascending colon, transverse colon, descending colon), or rectum. In certain embodiments, a microbial consortium can be administered in all regions of the gastrointestinal tract.

### Immunotherapies

In certain embodiments, the presently disclosed microbial consortia can be administered with an immunotherapy. In certain embodiments, the immunotherapy is an immune checkpoint inhibitor (ICI). Examples of ICIs include, but are not limited to, antagonists of the PD-1, PD L1, CTLA-4, BTLA, TIM3, LAG-3, TIGIT, B7-H3, VISTA, or ICOS pathways. In certain embodiments, the ICI comprises an anti-PD1 antibody, an anti-PD-L1 antibody, an anti-CTLA-4 antibody, an anti-BTLA antibody, an anti-TIM3 antibody, an anti-LAG-3 antibody, an anti-LAG-3 antibody, an anti-TIGIT antibody, an anti-B7-H3 antibody, an anti-VISTA antibody, an anti-ICOS antibody, or a combination thereof. In certain embodiments, the ICI comprises an anti-PD1 antibody and an anti-CTLA-4 antibody. In certain embodiments, the ICI is an anti-PD1 antibody, such as pembrolizumab, nivolumab, cemiplimab, dostarlimab, retifanlimab-dlwr, or tislelizumab. In certain embodiments, the ICI is an anti-PD-L1 antibody, such as atezolizumab, avelumab, or durvalumab. In certain embodiments, the ICI is an anti-CTL4 antibody, such as ipilimumab or tremelimumab.

### Combination Therapy

In certain embodiments, a Microbial Consortium disclosed herein (e.g., Consortia 1-7, each as set forth in Table 3) can be administered in combination with other agents. In certain embodiments, a Microbial Consortium disclosed herein (e.g., Consortia 1-7, each as set forth in Table 3) can be administered with an antimicrobial agent, an antifungal agent, an antiviral agent, an antiparasitic agent, a food, a drink, a probiotic, or a prebiotic. In certain embodiments, a Microbial Consortium disclosed herein (e.g., Consortia 1-7, each as set forth in Table 3) can be administered subsequent to administration of an antimicrobial agent, an antifungal agent, an antiviral agent, an antiparasitic agent or a prebiotic. In certain embodiments, administration may be sequential over a period of hours or days, or simultaneously.

For example, in certain non-limiting embodiments, a microbial consortium can be administered with, or pre-administered with, one or more antibacterial agent selected from fluoroquinolone antibiotics (ciprofloxacin, Levaquin, floxin, tequin, avelox, and norflox); cephalosporin antibiotics (cephalexin, cefuroxime, cefadroxil, cefazolin, cephalothin, cefaclor, cefamandole, cefoxitin, cefprozil, and ceftobiprole); penicillin antibiotics (amoxicillin, ampicillin, penicillin V, dicloxacillin, carbenicillin, vancomycin, and methicillin); tetracycline antibiotics (tetracycline, minocycline, oxytetracycline, and doxycycline); and carbapenem antibiotics (ertapenem, doripenem, imipenem/cilastatin, and meropenem).

For example, in certain non-limiting embodiments, a microbial consortium can be administered with one or more antiviral agent selected from Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuviltide, Etravirine, Famciclovir, Foscamet, Fomivirsen, Ganciclovir, Indinavir, Idoxuridine, Lamivudine, Lopinavir Maraviroc, MK-2048, Nelfinavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir, and Zidovudine.

In certain embodiments, a microbial consortium can be administered with one or more antifungal agent selected from miconazole, ketoconazole, clotrimazole, econazole, omoconazole, bifonazole, butoconazole, fenticonazole, isoconazole, oxiconazole, sertaconazole, sulconazole, and tioconazole; triazole antifungals such as fluconazole, itraconazole, isavuconazole, ravuconazole, posaconazole, voriconazok, terconazole, and albaconazole; thiazole antifungals such as abafungin; allylamine antifungals such as terbinafine, naftifine, and butenafine; and echinocandin antifungals such as anidulafungin, caspofungin, and micafungin; polygodial; benzoic acid; ciclopirox; tolnaftate; undecylenic acid; flucytosine or 5-fluorocytosine; griseofulvin; and haloprogin.

In certain embodiments, a microbial consortium can be administered with one or more anti-inflammatory and/or immunosuppressive agent selected from cyclophosphamide, mycophenolate mofetil, corticosteroids, mesalazine, mesalamine, sulfasalazine, sulfasalazine derivatives, cyclosporin A, mercaptopurine, azathiopurine, prednisone, methotrexate, antihistamines, glucocorticoids, epinephrine, theophylline, cromolyn sodium, anti-leukotrienes, anticholinergics, monoclonal anti-IgE, immunomodulatory peptides, immunomodulatory small molecules, immunomodulatory cytokines, immunomodulatory antibodies, and vaccines.

In certain embodiments, a Microbial Consortium disclosed herein (e.g., Microbial Consortium 1, Microbial Consortium 2, Microbial Consortium 3, Microbial Consortium 4, Microbial Consortium 5, Microbial Consortium 6, or Microbial Consortium 7) can be administered with one or more prebiotic selected from, but not limited to, amino acids, biotin, fructooligosaccharides, galactooligosaccharides, inulin, lactulose, mannan oligosaccharides, oligofructose-enriched inulin, oligofructose, oligodextrose, tagatose, trans-galacto-oligosaccharide, and xylooligosaccharides.

In certain embodiments, a Microbial Consortium disclosed herein (e.g., Consortia 1-7, each as set forth in Table 3) is administered in combination with an anti-inflammatory drug.

In certain embodiments, a Microbial Consortium disclosed herein (e.g., Consortia 1-7, each as set forth in Table 3) is administered in combination with an anti-diarrheal drug.

In certain embodiments, a Microbial Consortium disclosed herein (e.g., Consortia 1-7, each as set forth in Table 3) is administered in combination with a pain reliever. In certain embodiments, a Microbial Consortium disclosed herein (e.g., Consortia 1-7, each as set forth in Table 3) is administered in combination with a nonsteroidal anti-inflammatory drug (NSAIDs).

In certain embodiments, a Microbial Consortium disclosed herein (e.g., Consortia 1-7, each as set forth in Table 3) is administered in combination with vitamins and supplements.

In certain embodiments, a Microbial Consortium disclosed herein (e.g., Consortia 1-7, each as set forth in Table 3) is administered in combination with aminosalicylates.

In certain embodiments, a Microbial Consortium disclosed herein (e.g., Consortia 1-7, each as set forth in Table 3) is administered in combination with antibiotics.

In certain embodiments, a Microbial Consortium disclosed herein (e.g., Consortia 1-7, each as set forth in Table 3) is administered in combination with biologics. In certain embodiments, the biologics interrupt signals from the immune system that cause inflammation.

In certain embodiments, a Microbial Consortium disclosed herein (e.g., Consortia 1-7, each as set forth in Table 3) is administered in combination with corticosteroids or steroids.

In certain embodiments, a Microbial Consortium disclosed herein (e.g., Consortia 1-7, each as set forth in Table 3) is administered in combination with immunomodulators.

In certain embodiments, a Microbial Consortium disclosed herein (e.g., Consortia 1-7, each as set forth in Table 3) is administered in combination with any of the immune checkpoint inhibitors disclosed herein.

In certain embodiments, the combination treatment of a Microbial Consortium disclosed herein (e.g., Consortia 1-7, each as set forth in Table 3) comprises the pretreatment with antibiotics.

In certain embodiments, the pretreatment of antibiotics.

In certain embodiments, a bowel preparation (e.g., MiraLax) is administered in the late afternoon or early evening following the final dose of antibiotics.

### Pharmaceutical Compositions

The present disclosure also provides pharmaceutical compositions that contain an effective amount of a microbial consortium described herein. The composition can be formulated for use in a variety of delivery systems. One or more physiologically acceptable buffer(s) or carrier(s) can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In certain embodiments, the presently disclosed pharmaceutical composition comprises Consortia 1-7, each as set forth in Table 3.

In certain embodiments, the presently disclosed pharmaceutical composition comprises microbial consortia 7 of Table 3. The relevance of the microbial consortia 7 of Table 3 with respect to therapeutic effect is such that the composition may include those microbial species of consortia 7 of Table 3 as the only microbial species in the composition. Consequently, the presently disclosed pharmaceutical composition comprises microbial consortia comprising or consisting of Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica. Alternatively, the presently disclosed pharmaceutical composition comprises microbial consortia comprising or consisting of FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908.

In certain embodiments, the presently disclosed pharmaceutical composition comprises an amount of at least about 1, at least about 10, at least about 10², at least about 10³, at least about 10⁴, at least about 10⁵, at least about 10⁶, at least about 10⁷, at least about 10⁸, at least about 10⁹, at least about 10¹⁰, at least about 10¹¹, at least about 10¹², or at least about 10¹³ microbes of each strain (e.g., each strain of Consortia 7 as set forth in Table 3). In certain embodiments, the presently disclosed pharmaceutical composition comprises between about 1 and about 10¹³ between about 10 and about 10¹³, between about 10² and about 10¹³, between about 10³ and about 10¹³, between about 10⁴ and about 10¹³, between about 10⁵ and about 10¹³, between about 10⁶ and about 10¹³, between about 10⁷ and about 10¹³, between about 10⁸ and about 10¹³, between about 10⁹ and about 10¹³, between about 10¹⁰ and about 10¹³, between about 10¹¹ and about 10¹³, between about 10¹² and about 10¹³, between about 10 and about 10¹², between about 10 and about 10¹¹, between about 10 and about 10¹⁰, between about 10 and about 10⁹, between about 10 and about 10⁸, between about 10 and about 10⁷, between about 10 and about 10⁶, between about 10 and about 10⁵, between about 10 and about 10⁴, between about 10 and about 10³, between about 10² and about 10¹², between about 10² and about 10¹¹, between about 10² and about 10¹⁰, between about 10² and about 10⁹, between about 10² and about 10⁸, between about 10² and about 10⁷, between about 10² and about 10⁶, between about 10² and about 10⁵, between about 10² and about 10⁴, between about 10² and about 10³, between about 10² and about 10⁹, between about 10³ and about 10⁹, between about 10⁴ and about 10⁹, between about 10⁵ and about 10⁹, between about 10⁶ and about 10⁹, between about 10⁷ and about 10⁹, between about 10⁸ and about 10⁹, between about 10² and about 10⁶, between about 10³ and about 10⁶, between about 10⁴ and about 10⁶, or between about 10⁵ and about 10⁶ microbes for each strain (e.g., each strain of Consortia 7 as set forth in Table 3). In certain embodiments, each strain can be present in different amounts. For example, but without any limitation, a pharmaceutical composition comprising a first strain, a second strain, and a third strain can comprise 10 microbes of the first strain, 10⁷ microbes of the second strain, 10³ microbes of the third strain.

In certain embodiments, the presently disclosed pharmaceutical composition comprises an amount of at least about 10, at least about 10², at least about 10³, at least about 10⁴, at least about 10⁵, at least about 10⁶, at least about 10⁷, at least about 10⁸, at least about 10⁹, at least about 10¹⁰, at least about 10¹¹, at least about 10¹², or at least about 10¹³ CFUs of each strain (e.g., each strain of Consortia 7 as set forth in Table 3). In certain embodiments, the presently disclosed pharmaceutical composition comprises between about 1 and about 10¹³, about 10 and about 10¹³, between about 10² and about 10¹³, between about 10³ and about 10¹³, between about 10⁴ and about 10¹³, between about 10⁵ and about 10¹³, between about 10⁶ and about 10¹³, between about 10⁷ and about 10¹³, between about 10⁸ and about 10¹³, between about 10⁹ and about 10¹³, between about 10¹⁰ and about 10¹³, between about 10¹¹ and about 10¹³, between about 10¹² and about 10¹³, between about 10² and about 10¹², between about 10² and about 10¹¹, between about 10² and about 10¹⁰, between about 10² and about 10⁹, between about 10² and about 10⁸, between about 10² and about 10⁷, between about 10² and about 10⁶, between about 10² and about 10⁵, between about 10² and about 10⁴, between about 10² and about 10³, between about 10² and about 10⁹, between about 10³ and about 10⁹, between about 10⁴ and about 10⁹, between about 10⁵ and about 10⁹, between about 10⁶ and about 10⁹, between about 10⁷ and about 10⁹, between about 10⁸ and about 10⁹, between about 10² and about 10⁶, between about 10³ and about 10⁶, between about 10⁴ and about 10⁶, or between about 10⁵ and about 10⁶ CFUs for each strain (e.g., each strain of Consortia 7 as set forth in Table 3). In certain embodiments, each strain can be present in different amounts. For example, but without any limitation, a pharmaceutical composition comprising a first strain, a second strain, and a third strain can comprise 10 CFUs of the first strain, 10⁷ CFUs of the second strain, 10³ CFUs of the third strain.

In certain embodiments, the presently disclosed pharmaceutical composition comprises an amount of at least about 101, at least about 10⁻², at least about 10⁻³, at least about 10⁻⁴, at least about 10⁻⁵, at least about 10⁻⁶, at least about 10⁻⁷, at least about 10⁻⁸, at least about 10⁻⁹, at least about 10⁻¹⁰, at least about 10⁻¹¹, or at least about 10⁻¹², or at least about 10¹³ grams of each strain (e.g., each strain of Consortia 7 as set forth in Table 3). In certain embodiments, each strain can be present in different amounts. For example, but without any limitation, a pharmaceutical composition comprising a first strain, a second strain, and a third strain can comprise 10⁻⁴ grams of the first strain, 10⁻³ grams of the second strain, 10⁻⁶ grams of the third strain.

In certain embodiments, the presently disclosed pharmaceutical composition comprises a total amount of at least about 1, at least about 10, at least about 10², at least about 10³, at least about 10⁴, at least about 10⁵, at least about 10⁶, at least about 10⁷, at least about 10⁸, at least about 10⁹, at least about 10¹⁰, at least about 10¹¹, at least about 10¹², or at least about 10¹³ microbes (e.g., total amount of microbes of Consortia 7 as set forth in Table 3). In certain embodiments, the presently disclosed pharmaceutical composition comprises a total amount of between about 10 and about 10¹³, between about 10² and about 10¹³, between about 10³ and about 10¹³, between about 10⁴ and about 10¹³, between about 10⁵ and about 10¹³, between about 10⁶ and about 10¹³, between about 10⁷ and about 10¹³, between about 10⁸ and about 10¹³, between about 10⁹ and about 10¹³, between about 10¹⁰ and about 10¹³, between about 10¹¹ and about 10¹³, between about 10¹² and about 10¹³, between about 10² and about 10¹², between about 10² and about 10¹¹, between about 10² and about 10¹⁰, between about 10² and about 10⁹, between about 10² and about 10⁸, between about 10² and about 10⁷, between about 10² and about 10⁶, between about 10² and about 10⁵, between about 10² and about 10⁴, between about 10² and about 10³, between about 10² and about 10⁹, between about 10³ and about 10⁹, between about 10⁴ and about 10⁹, between about 10⁵ and about 10⁹, between about 10⁶ and about 10⁹, between about 10⁷ and about 10⁹, between about 10⁸ and about 10⁹, between about 10² and about 10⁶, between about 10³ and about 10⁶, between about 10⁴ and about 10⁶, or between about 10⁵ and about 10⁶ microbes.

In certain embodiments, the presently disclosed pharmaceutical composition comprises a total amount of at least about 1, at least about 10, at least about 10², at least about 10³, at least about 10⁴, at least about 10⁵, at least about 10⁶, at least about 10⁷, at least about 10⁸, at least about 10⁹, at least about 10¹⁰, at least about 10¹¹, at least about 10¹², or at least about 10¹³ CFUs (e.g., total CFUs of Consortia 7 as set forth in Table 3). In certain embodiments, the presently disclosed pharmaceutical composition comprises a total amount of between about 10 and about 10¹³, between about 10² and about 10¹³, between about 10³ and about 10¹³, between about 10⁴ and about 10¹³, between about 10⁵ and about 10¹³, between about 10⁶ and about 10¹³, between about 10⁷ and about 10¹³, between about 10⁸ and about 10¹³, between about 10⁹ and about 10¹³, between about 10¹⁰ and about 10¹³, between about 10¹¹ and about 10¹³, between about 10¹² and about 10¹³, between about 10² and about 10¹², between about 10² and about 10¹¹, between about 10² and about 10¹⁰, between about 10² and about 10⁹, between about 10² and about 10⁸, between about 10² and about 10⁷, between about 10² and about 10⁶, between about 10² and about 10⁵, between about 10² and about 10⁴, between about 10² and about 10³, between about 10² and about 10⁹, between about 10³ and about 10⁹, between about 10⁴ and about 10⁹, between about 10⁵ and about 10⁹, between about 10⁶ and about 10⁹, between about 10⁷ and about 10⁹, between about 10⁸ and about 10⁹, between about 10² and about 10⁶, between about 10³ and about 10⁶, between about 10⁴ and about 10⁶, or between about 10⁵ and about 10⁶ CFUs. In certain embodiments, the presently disclosed pharmaceutical composition comprises between about 10⁵ and about 10¹³, between about 10⁶ and about 10¹³, between about 107 and about 10¹³, between about 10⁸ and about 10¹³, between about 10⁹ and about 10¹³, between about 10¹⁰ and about 10¹³, between about 10¹¹ and about 10¹³, between about 10¹² and about 10¹³, between about 10⁵ and about 10¹², between about 10⁶ and about 10¹², between about 10⁷ and about 10¹², between about 10⁸ and about 10¹², between about 10⁹ and about 10¹², between about 10¹⁰ and about 10¹², between about 10¹¹ and about 10¹², between about 10⁵ and about 10¹¹, between about 10⁶ and about 10¹¹, between about 10⁷ and about 10¹¹, between about 10⁸ and about 10¹¹, between about 10⁹ and about 10¹¹, between about 10¹⁰ and about 10¹¹, between about 10⁵ and about 10¹⁰, between about 10⁶ and about 10¹⁰, between about 10⁷ and about 10¹⁰, between about 10⁸ and about 10¹⁰, between about 10⁹ and about 10¹⁰, between about 10⁵ and about 10⁹, between about 10⁶ and about 10⁹, between about 10⁷ and about 10⁹, between about 10⁸ and about 10⁹, between about 5×10⁹ and about 5×10¹⁰, between about 5×10⁹ and about 5×10¹¹, between about 5×10⁹ and about 5×10¹², between about 5×10¹⁰ and about 5×10¹², between about 5×10¹¹ and about 5×10¹², or between about 5×10¹⁰ and about 5×10¹¹ viable cells. In certain embodiments, the presently disclosed pharmaceutical composition comprises between about 5×10⁹ and about 5×10¹² viable cells. In certain embodiments, the presently disclosed pharmaceutical composition comprises between about 5×10⁹ and about 5×10¹⁰ viable cells. In certain embodiments, the presently disclosed pharmaceutical composition comprises between about 5×10¹⁰ and about 5×10¹¹ viable cells. In certain embodiments, the presently disclosed pharmaceutical composition comprises between about 5×10¹¹ and about 5×10¹² viable cells.

In certain embodiments, the presently disclosed pharmaceutical composition comprises up to about 10⁵, up to about 10⁶, up to about 10⁷, up to about 10⁸, up to about 10⁹, up to about 10¹⁰, up to about 10¹¹, up to about 10¹², or up to about 10¹³ viable cells. In certain embodiments, the presently disclosed pharmaceutical composition comprises up to about 10¹¹ viable cells. In certain embodiments, the presently disclosed pharmaceutical composition comprises up to about 10¹² viable cells.

In certain embodiments, the presently disclosed pharmaceutical composition can be present in the form of a food product comprising a consortium disclosed herein (e.g., Consortia 7 as set forth in Table 3). As used herein, the term “food product” refers to a composition intended for ingestion by an individual (e.g., a human subject). Non-limiting examples of food products encompassed by the present disclosure include juices, refreshing beverages, tea beverages, drink preparations, jelly beverages, functional beverages, milk, dairy beverages, ice creams, cheeses, yogurts, biscuits, cookies, candies, chewing gums, gummies, jellies, cream caramels, frozen desserts, and instant foods. Further, the examples also include health foods and beverages prepared in the forms of powders, granules, tablets, capsules, liquids, pastes, and jellies. In certain embodiments, the food product comprising a presently disclosed consortia further comprises a prebiotic. As used herein, the term “prebiotic” refers to a substance that can promote the growth of the microbes of the consortia. Non-limiting examples of prebiotic include fructose, galactose, mannose, soy, inulin, dietary fibers, or a combination thereof.

In certain embodiments, microbial cells of the present disclosure are harvested by microfiltration and centrifugation. In certain embodiments, microfiltration is done with a membrane comprising a nonreactive polymer. For example, in certain non-limiting embodiments, said membrane comprises Polyvinylidene fluoride, Polysulfones, or nitrocellulose. In certain embodiments, a membrane for microfiltration has a pore size from about 0.2 μm to about 0.45 μm. In certain embodiments, the cells are centrifuged at from about 1000 g to about 30000 g, from about 5000 g to about 30000 g, from about 10000 g to about 30000 g, from about 15000 g to about 30000 g, from about 20000 g to about 30000 g, from about 25000 g to about 30000 g, from about 1000 g to about 25000 g, from about 5000 g to about 25000 g, from about 10000 g to about 25000 g, from about 15000 g to about 25000 g, from about 20000 g to about 25000 g, from about 1000 g to about 20000 g, from about 5000 g to about 20000 g, from about 10000 g to about 20000 g, from about 15000 g to about 20000 g, from about 1000 g to about 15000 g, from about 5000 g to about 15000 g, from about 10000 g to about 15000 g, from about 1000 g to about 10000 g, from about 5000 g to about 10000 g, or from about 1000 g to about 5000 g force.

In certain embodiments, the cells are concentrated to from about 1×10⁶ CFUs per milliliter to about 1×10¹² CFUs per milliliter, from about 1×10⁷ CFUs per milliliter to about 1×10¹² CFUs per milliliter, from about 1×10⁸ CFUs per milliliter to about 1×10¹² CFUs per milliliter, from about 1×10⁹ CFUs per milliliter to about 1×10¹² CFUs per milliliter, from about 1×10¹⁰ CFUs per milliliter to about 1×10¹² CFUs per milliliter, from about 1×10¹¹ CFUs per milliliter to about 1×10¹² CFUs per milliliter, from about 1×10⁶ CFUs per milliliter to about 1×10¹¹ CFUs per milliliter, from about 1×10⁷ CFUs per milliliter to about 1×10¹¹ CFUs per milliliter, from about 1×10⁸ CFUs per milliliter to about 1×10¹¹ CFUs per milliliter, from about 1×10⁹ CFUs per milliliter to about 1×10¹¹ CFUs per milliliter, from about 1×10¹⁰ CFUs per milliliter to about 1×10¹¹ CFUs per milliliter, from about 1×10⁶ CFUs per milliliter to about 1×10¹⁰ CFUs per milliliter, from about 1×10⁷ CFUs per milliliter to about 1×10¹⁰ CFUs per milliliter, from about 1×10⁸ CFUs per milliliter to about 1×10¹⁰ CFUs per milliliter, from about 1×10⁹ CFUs per milliliter to about 1×10¹⁰ CFUs per milliliter, from about 1×10⁶ CFUs per milliliter to about 1×10⁹ CFUs per milliliter, from about 1×10⁷ CFUs per milliliter to about 1×10⁹ CFUs per milliliter, from about 1×10⁸ CFUs per milliliter to about 1×10⁹ CFUs per milliliter, from about 1×10⁶ CFUs per milliliter to about 1×10⁸ CFUs per milliliter, from about 1×10⁷ CFUs per milliliter to about 1×10⁸ CFUs per milliliter, or from about 1×10⁶ CFUs per milliliter to about 1×10⁷ CFUs per milliliter.

In certain embodiments, microbial cells of the present disclosure are frozen. In certain embodiments, the microbial cells of the present disclosure are mixed with one or more cryoprotective agents (CPAs) before freezing. In certain embodiments, the ratio of cells to CPA is approximately 25:1, 10:1, 5:1, 4:1, 3:1, 2:1, 1:1, 1:2, 1:3, 1:4, 1:5, 1:10, or 1:25. In certain embodiments, a CPA comprises one or more of glycerol, maltodextrin, sucrose, inulin, trehalose, and alginate. In certain embodiments, a CPA further comprises one or more antioxidants. In certain embodiments, an antioxidant is selected from the list of cysteine, ascorbic acid, and riboflavin.

### Dosages

Dosing and schedule can include a larger loading dose at 1e10, 1e11, 1e12, or up to 1e13 cells. Dosing can be one-time or include maintenance dosing. Maintenance dose can be 1e9, 1e10, or 1e11 cells, dosed multiple times per day, daily, weekly, monthly, or quarterly.

In certain embodiments, a microbial consortium is administered in a dosage form having a total amount of microbial consortium of 0.1 ng to 500 mg, 0.5 ng to 500 mg, 1 ng to 500 mg, 5 ng to 500 mg, 10 ng to 500 mg, 50 ng to 500 mg, 100 ng to 500 mg, 500 ng to 500 mg, 1 μg to 500 mg, 5 μg to 500 mg, 10 μg to 500 mg, 50 μg to 500 mg, 100 μg to 500 mg, 500 μg to 500 mg, 1 mg to 500 mg, 5 mg to 500 mg, 10 mg to 500 mg, 50 mg to 500 mg, 100 mg to 500 mg, 0.1 ng to 100 mg, 0.5 ng to 100 mg, 1 ng to 100 mg, 5 ng to 100 mg, 10 ng to 100 mg, 50 ng to 100 mg, 100 ng to 100 mg, 500 ng to 500 mg, 1 μg to 100 mg, 5 μg to 100 mg, 10 μg to 100 mg, 50 μg to 100 mg, 100 μg to 100 mg, 500 μg to 100 mg, 1 mg to 500 mg, 5 mg to 100 mg, 10 mg to 100 mg, 50 mg to 100 mg, 0.1 ng to 50 mg, 0.5 ng to 50 mg, 1 ng to 50 mg, 5 ng to 50 mg, 10 ng to 50 mg, 50 ng to 50 mg, 100 ng to 50 mg, 500 ng to 500 mg, 1 μg to 50 mg, 5 μg to 50 mg, 10 μg to 50 mg, 50 μg to 50 mg, 100 μg to 50 mg, 500 μg to 50 mg, 1 mg to 500 mg, 5 mg to 50 mg, 10 mg to 50 mg, 0.1 ng to 10 mg, 0.5 ng to 10 mg, 1 ng to 10 mg, 5 ng to 10 mg, 10 ng to 10 mg, 50 ng to 10 mg, 100 ng to 10 mg, 500 ng to 500 mg, 1 μg to 10 mg, 5 μg to 10 mg, 10 μg to 10 mg, 50 μg to 10 mg, 100 μg to 10 mg, 500 μg to 10 mg, 1 mg to 500 mg, 5 mg to 10 mg, 0.1 ng to 5 mg, 0.5 ng to 5 mg, 1 ng to 5 mg, 5 ng to 5 mg, 10 ng to 5 mg, 50 ng to 5 mg, 100 ng to 5 mg, 500 ng to 500 mg, 1 μg to 5 mg, 5 μg to 5 mg, 10 μg to 5 mg, 50 μg to 5 mg, 100 μg to 5 mg, 500 μg to 5 mg, 1 mg to 500 mg, 0.1 ng to 1 mg, 0.5 ng to 1 mg, 1 ng to 1 mg, 5 ng to 1 mg, 10 ng to 1 mg, 50 ng to 1 mg, 100 ng to 1 mg, 500 ng to 500 mg, 1 μg to 1 mg, 5 μg to 1 mg, 10 μg to 1 mg, 50 μg to 1 mg, 100 μg to 1 mg, 500 μg to 1 mg, 0.1 ng to 500 μg, 0.5 ng to 500 μg, 1 ng to 500 μg, 5 ng to 500 μg, 10 ng to 500 μg, 50 ng to 500 μg, 100 ng to 500 μg, 500 ng to 500 μg, 1 μg to 500 μg, 5 μg to 500 μg, 10 μg to 500 μg, 50 μg to 500 μg, 100 μg to 500 μg, 0.1 ng to 100 μg, 0.5 ng to 100 μg, 1 ng to 100 μg, 5 ng to 100 μg, 10 ng to 100 μg, 50 ng to 100 μg, 100 ng to 100 μg, 500 ng to 100 μg, 1 μg to 100 μg, 5 μg to 100 μg, 10 μg to 100 μg, 50 μg to 100 μg, 0.1 ng to 50 μg, 0.5 ng to 50 μg, 1 ng to 50 lag, 5 ng to 50 μg, 10 ng to 50 μg, 50 ng to 50 μg, 100 ng to 50 μg, 500 ng to 50 g, 1 μg to 50 g, 5 μg to 50 g, 10 μg to 50 g, 0.1 ng to 10 μg, 0.5 ng to 10 μg, 1 ng to 10 μg, 5 ng to 10 μg, 10 ng to 10 μg, 50 ng to 10 μg, 100 ng to 10 μg, 500 ng to 10 μg, 1 μg to 10 μg, 5 μg to 10 μg, 0.1 ng to 5 μg, 0.5 ng to 5p g, 1 ng to 5p g, 5 ng to 5 μg, 10 ng to 5 μg, 50 ng to 5 μg, 100 ng to 5 μg, 500 ng to 5 μg, 1 μg to 5 μg, 0.1 ng to 1 μg, 0.5 ng to 1 μg, 1 ng to 1 μg, 5 ng to 1 μg, 10 ng to 1 μg, 50 ng to 1 μg, 100 ng to 1 μg, 500 ng to 1 μg, 0.1 ng to 500 ng, 0.5 ng to 500 ng, 1 ng to 500 ng, 5 ng to 500 ng, 10 ng to 500 ng, 50 ng to 500 ng, 100 ng to 500 ng, 0.1 ng to 100 ng, 0.5 ng to 100 ng, 1 ng to 100 ng, 5 ng to 100 ng, 10 ng to 100 ng, 50 ng to 100 ng, 0.1 ng to 50 ng, 0.5 ng to 50 ng, 1 ng to 50 ng, 5 ng to 50 ng, 10 ng to 50 ng, 0.1 ng to 10 ng, 0.5 ng to 10 ng, 1 ng to 10 ng, 5 ng to 10 ng, 0.1 ng to 5 ng, 0.5 ng to 5 ng, 1 ng to 5 ng, 0.1 ng to 1 ng, 0.1 ng to 1 ng, or 0.1 ng to 0.5 ng total dry weight.

In certain embodiments, a microbial consortium is administered as a single dose or as multiple doses. In certain embodiments, a microbial consortium is administered intermittently. For example, in certain non-limiting embodiments, a microbial consortium is administered once weekly, once monthly, or when a subject is in need thereof. In certain embodiments, the consortium is administered once a day for 2 days, 3 days, 4 days, 5 days, 6 days, or 1 week. In certain embodiments, the consortium is administered once a week for 2 weeks, 3 weeks, or 1 month. In some embodiments, the consortium is administered once a month for 2 months, 3 months, 4 months, 5 months, 6 months, or 1, 2, 3, or more years.

In certain embodiments, the consortium is administered at an effective dose to allow for engraftment.

### Formulation: Drug Substance Formulation

After co-culture fermentation, the microbial biomass can be harvested, concentrated, and formulated, e.g., with glycerol (10-25%) before being stored frozen at ≤−65° C. Lyophilization can also be employed.

### Formulation: Drug Product Formulation

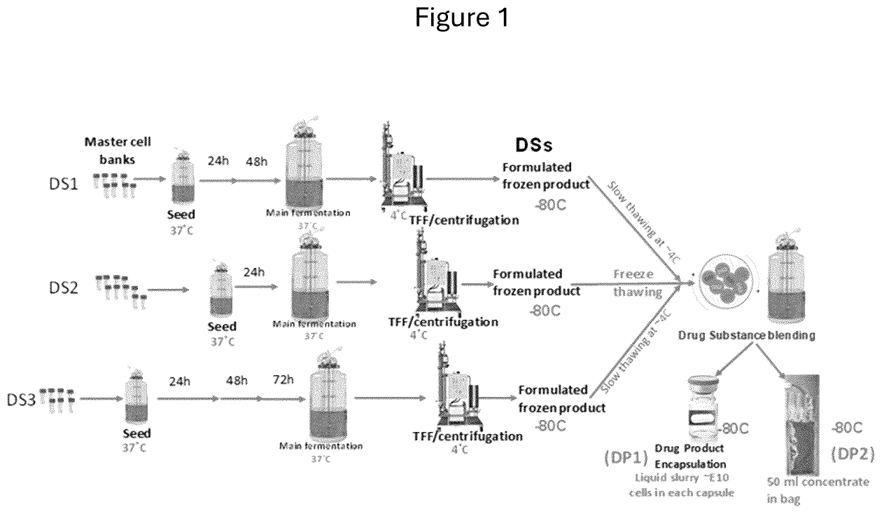

For drug product formulation, a drug substance can be thawed and blended before manual filling into capsules (DP1) or freezer-stable bags (DP2) or lyophilized and put into capsules (DP3). The filled capsules and bags will be stored at ≤−65° C. within a container-closure system. Further detail is provided below and in FIG. 1.
DP1 for oral route: The blended drug substance material can be filled into gelatin or capsules.
DP2 for colonoscopy route: The blended drug substance material can be filled into freezer-stable bags.

Colonoscopic delivery of frozen material has demonstrated success clinically. Treatment can involve this route, with optional oral maintenance. Oral delivery is also a possibility.

If engraftment is not successful, future efforts could involve oral delivery and possible antibiotic pre-treatment.

### Methods of Preparation, Isolation, and Culture

The present disclosure also provides methods for preparing and/or manufacturing the microbial consortia described herein.

In certain embodiments, the methods comprise obtaining a donor stool and preparing a stool dilution. In certain embodiments, the stool dilution is plated onto an agar plate. In certain embodiments, the agar plate includes an anaerobic media. In certain embodiments, the agar plate includes colonies. Characterization and quality analysis of these colonies can be performed. For example, but without any limitation, 16s RNA and/or MALDI mass spectrometry could be performed. In certain embodiments, the characterized colonies can be further expanded in a broth culture. After growth and expansion, the microbes can be stored in vials for further use.

In certain embodiments, the microbes can be further expanded in a bioreactor including a cell culture medium. In certain embodiments, the cell culture medium can include:
a) soytone, D-cellobiose, yeast extract, dextrose (glucose), maltose monohydrate, magnesium sulfate heptahydrate, calcium chloride dihydrate, potassium phosphate monobasic, potassium phosphate dibasic, sodium chloride, sodium bicarbonate, volatile fatty acid solution, L-cysteine HCl monohydrate, hemin solution, vitamin solution, or a combination thereof, or
b) soytone, D-cellobiose, yeast extract, dextrose (glucose), maltose monohydrate, magnesium sulfate heptahydrate, calcium chloride dihydrate, potassium phosphate monobasic, potassium phosphate dibasic, sodium chloride, ammonium sulfate, sodium bicarbonate, volatile fatty acid solution, L-cysteine HCl monohydrate, hemin solution, vitamin solution, or a combination thereof.

In certain embodiments, the cell culture medium is YCFAC. In certain embodiments, the cell culture medium further comprises threonine. In certain embodiments, the cell culture media is YSFAC.

In certain embodiments, the microbes can be expanded in a bioreactor in anaerobic conditions. In certain embodiments, the microbes can be expanded in a bioreactor in the presence of gas overlay. In certain embodiments, the microbes can be expanded in a bioreactor in absence of gas sparing.

In certain embodiments, the methods include expanding microbes in mixed cultures.

In certain embodiments, the methods comprise lyophilizing cultures and compositions described herein. In certain embodiments, the cultures and compositions comprise a lyoprotectant.

In certain embodiments, the lyoprotectant comprises maltodextrin. In certain embodiments, the lyoprotectant comprises inulin. In certain embodiments, the lyoprotectant comprises maltodextrin and inulin. In certain embodiments, the maltodextrin is present at a concentration of about 8%. In certain embodiments, the inulin is present at a concentration of about 0.5%.

In certain embodiments, the presently disclosed microbial consortia are prepared using the methods described in the Example section.

Methods for bacterial isolation and culture of the microbial consortia of the present disclosure are provided in the Examples. Additionally, further methods for isolation are described, for example, in U.S. Provisional Patent Application No. 63/716,534, filed Nov. 5, 2024, which is incorporated by reference in its entirety. Additional culture methods are disclosed, for example, in patent application publications WO2021/183701 and US2023/0165913, each of which is incorporated by reference in its entirety.

### Kits

The presently disclosed subject matter provides kits for treating or preventing a cancer in a subject in need thereof. In certain embodiments, the subject has received or will receive an immunotherapy including an immune checkpoint inhibitor (ICI). In certain embodiments, the kit comprises an effective amount of a presently disclosed microbial consortium or a pharmaceutical composition comprising thereof.

In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes anaerobic containers to hold the microbial consortium described herein. In certain non-limiting embodiments, the kit includes blister packs to hold the microbial consortium described herein in the presence of no or limited amounts of oxygen. In certain non-limiting embodiments, the kit includes blister packs with desiccant to hold the microbial consortium described herein in the presence of no or limited amounts of oxygen. In certain non-limiting embodiments, the kit includes bottles with desiccant to hold the microbial consortium described herein in the presence of no or limited amounts of oxygen.

In certain embodiments, the kits include instructions for administering the microbial consortium as described herein. In certain embodiments, the instructions include directions for administering the loading and the maintenance dose.

In certain embodiments, the kits include storage instructions. In certain embodiments, the storage instructions are for storage at approximately −20° C. In certain embodiments, the storage instructions are for storage at less than −5° C. In certain embodiments, the storage instructions are for storage at less than approximately −15 to −20° C., −10 to −20° C., −10 to −15° C., −5 to −10° C., 0 to −5° C., below 0° C., or 0 to −20° C. In certain embodiments, the storage instructions are for storage at less than approximately 4° C. In certain embodiments, the storage instructions are for storage at room temperature.

In certain embodiments, the kits include instructions for maintaining the microbial consortium in no or low oxygen conditions.

In certain embodiments, the kits include instructions for the subject to remain off all antibiotics during treatment with the microbial consortium.

In certain embodiments, the kit includes microbial consortium and instructions for administering the same.

### EXAMPLES

The disclosure now being generally described, will be more readily understood by reference to the following examples which are included merely for purposes of illustration of certain aspects and embodiments of the present disclosure and are not intended to limit the scope of the disclosure in any way.

### Example 1: Super Donor Fecal Material Source

Fecal material was collected from a super donor who is a cancer survivor with a complete response to checkpoint immunotherapy (pembrolizumab). The super donor has been in remission since July 2022. FMT from the super donor demonstrated the ability to induce a therapeutic response to immunotherapy in previously non-responsive cancer patients. Fecal material was collected from the same super donor at multiple time points.

### Example 2: Species Isolation

The isolation of species from the super donor fecal material was conducted using a conventional method, matrix-assisted laser desorption ionization (MALDI) based liquid culture isolation, and with targeted liquid culture isolation. The material was quickly processed and stored under anaerobic conditions to preserve anaerobic species viability. The material was collected from three timepoints in order to compile a complete collection of species since individual species abundance may wax and wane over time.

The list of isolates from the super donor fecal material is provided in Tables 1 and 3. Also included are the sequence identifiers for the genomic sequences of the species.

### Example 3: Species Isolation, Stability, Growth Curves and Genomic Characterization

Further information on the particular methods of isolation and details on super donor source for each species are provided in Table 2. Details on methodology are provided in Example 8. For Isolation Method in Table 2, a designation of 1 is for the conventional method, 2 is for the MALDI-based liquid culture isolation, and 3 is for the targeted liquid culture isolation method.

TABLE 2
Isolation, Super Donor Source Donation Date and Isolation Medium

| | | SD Source | |
|---|---|---|---|
| Bacteria | Isolation | Donation | |
| ID | Method | Time Point | Isolation Medium (Agar) |
| FBI01839 | 1 | A | Anaerobe Systems BHI |
| FBI01840 | 1 | A | Anaerobe Systems BHI |
| FBI01848 | 1 | A | Anaerobe Systems BHI |
| FBI01859 | 1 | A | Anaerobe Systems BHI |
| FBI01861 | 1 | A | Anaerobe Systems BHI |
| FBI01863 | 1 | A | Anaerobe Systems BHI |
| FBI01866 | 1 | A | Anaerobe Systems BHI |
| FBI01876 | 1 | A | Anaerobe Systems BHI |
| FBI01883 | 1 | A | Anaerobe Systems BHI |
| FBI01900 | 1 | A | Anaerobe Systems BHI |
| FBI01907 | 1 | A | Anaerobe Systems BHI |
| FBI01925 | 1 | A | Anaerobe Systems BHI |
| FBI01930 | 1 | A | Anaerobe Systems BHI |
| FBI01952 | 1 | A | Anaerobe Systems BHI |
| FBI01956 | 1 | A | Anaerobe Systems BHI |
| FBI01957 | 1 | A | Anaerobe Systems BHI |
| FBI02069 | 1 | A | Anaerobe Systems BHI |
| FBI02074 | 1 | A | Anaerobe Systems BHI |
| FBI02083 | 1 | A | Anaerobe Systems BHI |
| FBI02101 | 1 | A | Anaerobe Systems MRS |
| FBI02106 | 1 | A | Anaerobe Systems LMRS |
| FBI02111 | 1 | A | Anaerobe Systems LMRS |
| FBI02144 | 1 | A | Anaerobe Systems LMRS |
| FBI02158 | 1 | A | Anaerobe Systems RCA |
| FBI02176 | 1 | A | Anaerobe Systems RCA |
| FBI02208 | 1 | A | Anaerobe Systems RCA |
| FBI02271 | 1 | A | Anaerobe Systems CDC |
| FBI02288 | 1 | A | Anaerobe Systems CDC |
| FBI02305 | 1 | A | Anaerobe Systems CDC |
| FBI02325 | 1 | A | Anaerobe Systems CDC |
| FBI02339 | 1 | A | Anaerobe Systems CDC |
| FBI02365 | 1 | A | Anaerobe Systems CDC |
| FBI02408 | 1 | A | Anaerobe Systems CDC |
| FBI02412 | 1 | A | Anaerobe Systems CDC |
| FBI02417 | 1 | A | Anaerobe Systems CDC |
| FBI02538 | 1 | A | BHI-mucin |
| FBI02555 | 1 | A | BHI-mucin |
| FBI02593 | 1 | A | BHI-mucin |
| FBI02695 | 1 | A | BHI-mucin |
| FBI02882 | 2 | B | Anaerobe Systems YCFAC |
| FBI02884 | 2 | B | Anaerobe Systems YCFAC |
| FBI02887 | 2 | B | Anaerobe Systems YCFAC |
| FBI02888 | 2 | B | Anaerobe Systems YCFAC |
| FBI02890 | 2 | A | Anaerobe Systems YCFAC |
| FBI02891 | 2 | A | Anaerobe Systems YCFAC |
| FBI02893 | 2 | B | Anaerobe Systems YCFAC |
| FBI02894 | 2 | B | Anaerobe Systems YCFAC |
| FBI02895 | 2 | A | Anaerobe Systems YCFAC |
| FBI02896 | 2 | A | Anaerobe Systems YCFAC |
| FBI02897 | 2 | A | Anaerobe Systems YCFAC |
| FBI02898 | 2 | A | Teknova BHI |
| FBI02899 | 2 | C | Anaerobe Systems YCFAC + |
| | | | Hemin/Vitamin K |
| FBI02900 | 2 | C | Anaerobe Systems YCFAC + |
| | | | Hemin/Vitamin K |
| FBI02901 | 2 | C | Anaerobe Systems YCFAC + |
| | | | Hemin/Vitamin K |
| FBI02902 | 2 | C | Anaerobe Systems YCFAC + |
| | | | Hemin/Vitamin K |
| FBI02903 | 2 | C | Anaerobe Systems YCFAC + |
| | | | Hemin/Vitamin K |
| FBI02904 | 3 | C | Anaerobe Systems YCFAC + |
| | | | Hemin/Vitamin K |
| FBI02905 | 3 | C | Anaerobe Systems YCFAC + |
| | | | Hemin/Vitamin K |
| FBI02906 | 3 | C | Anaerobe Systems YCFAC (50%) + |
| | | | Biochemazone Artificial Intestinal |
| | | | Fluid (50%) |
| FBI02908 | 2 | B | Anaerobe Systems YCFAC + 0.01% |
| | | | Tween |

Bacteria stability was assessed for each bacterial species after being frozen at −80° C. for ≥30 days using a colony forming unit (CFU) measurement. The species failed the stability assessment if there was a loss of more than 90% of viable cells (by CFU) after being thawed at the evaluation timepoint. All species passed the stability test except for Blautia wexlerae FBI01866 and Agathobacter rectalis FBI02901.

Growth curve data for each individual species was generated, measured using a dilution curve.

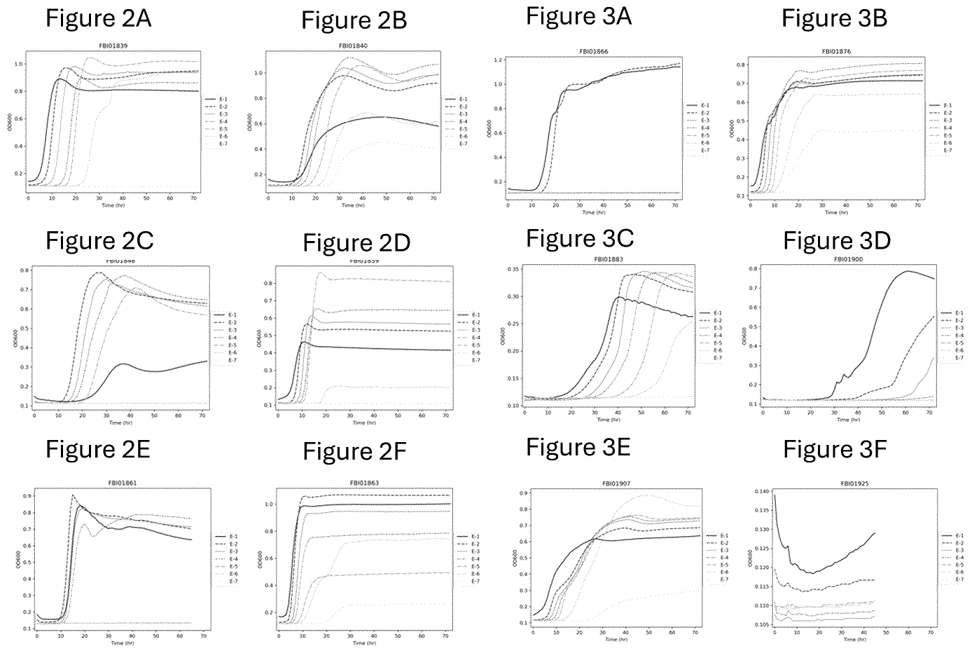

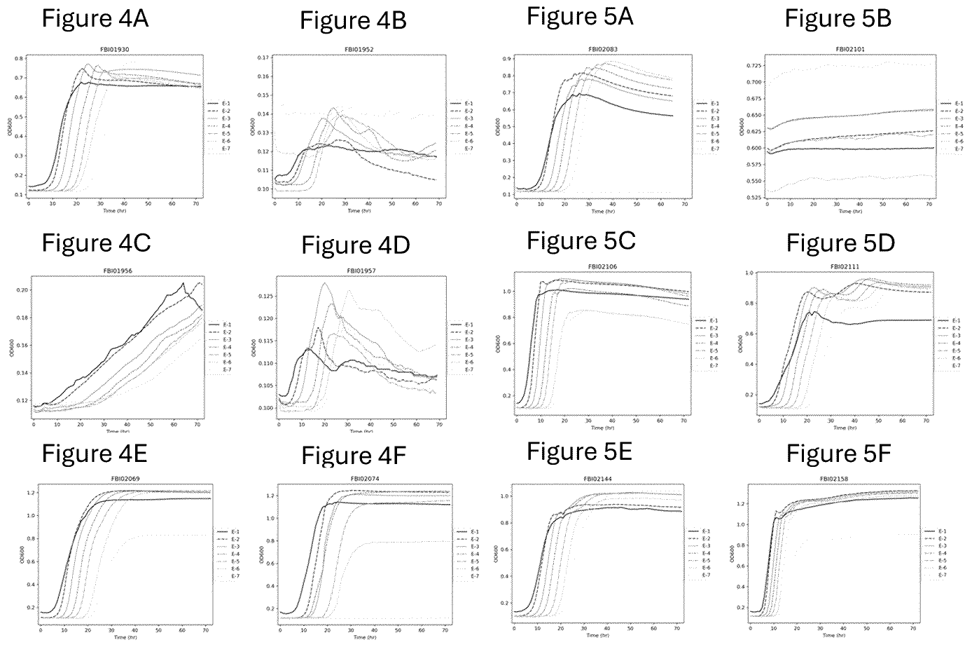

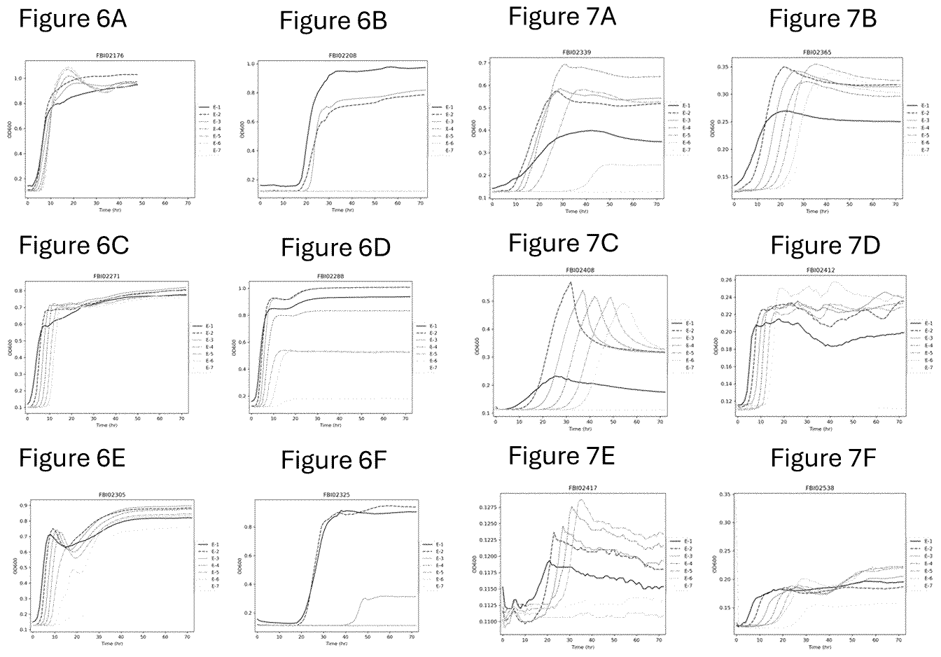

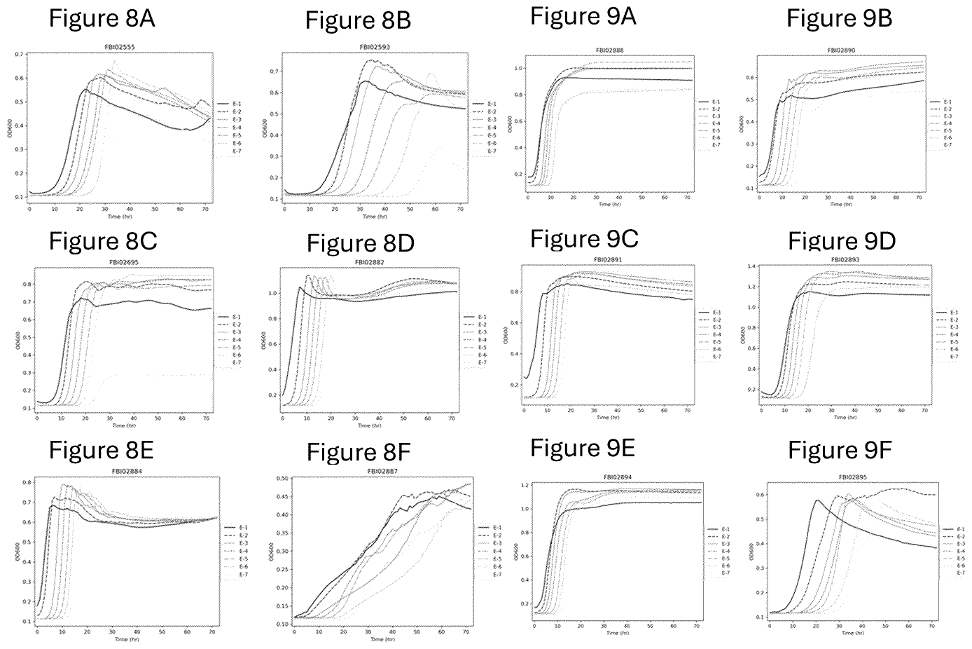

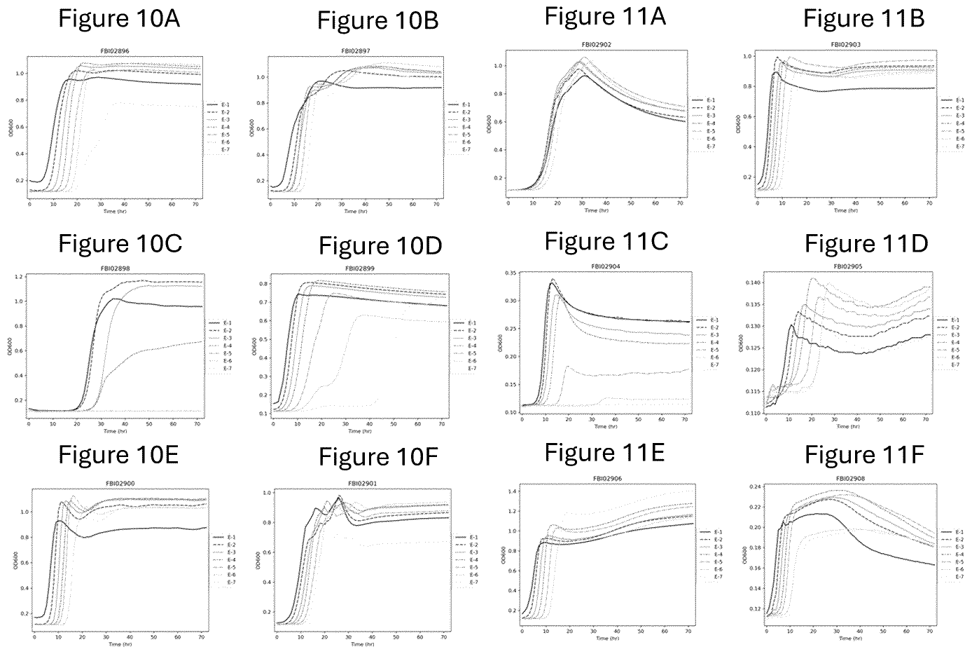

Data on growth curves are shown in FIGS. 2A-2F, 3A-3F, 4A-4F, 5A-5F, 5A-6F, 7A-7F, 8A-8F, 9A-9F, 10A-10F, and 11A-11F. When selecting bacterial species for microbial consortia, it may be beneficial for them to demonstrate similar growth properties as detailed here; however, the disclosed growth curves are not necessary to make each of the consortia described herein.

Screening for virulence factors and identification of non-typical gut commensals or potential opportunistic pathobionts identified led to exclusion of some species from some versions of the microbial consortia. Streptococcus and Enterococcus species were excluded from some microbial consortia versions.

Identification of species/strain specific signatures enabled detection in clinical sample.

### Example 4: Microbial Consortia Bacteria

Seven microbial consortia (Consortia 1, 2, 3, 4, 5, 6, and 7, each as set forth in Table 3) were generated as different subsets of the bacteria identified in the super donor fecal material. Versions 5.1, 6.1, and 6.2 were also designed in the process of consortia selection and optimization. The super donor species are listed as “Species” in Table 3 (and also in Table 1). Table 3 below also details the species makeup of each of the six microbial consortia. “Yes” denotes that the bacteria is present in that microbial consortium and a blacked-out box denotes that the bacteria was excluded from an individual consortium.

### Example 5: Microbial Consortia and Tumor Volume

The ability of the bacterial of the microbial consortia to induce control over tumor volume was assessed in various models.

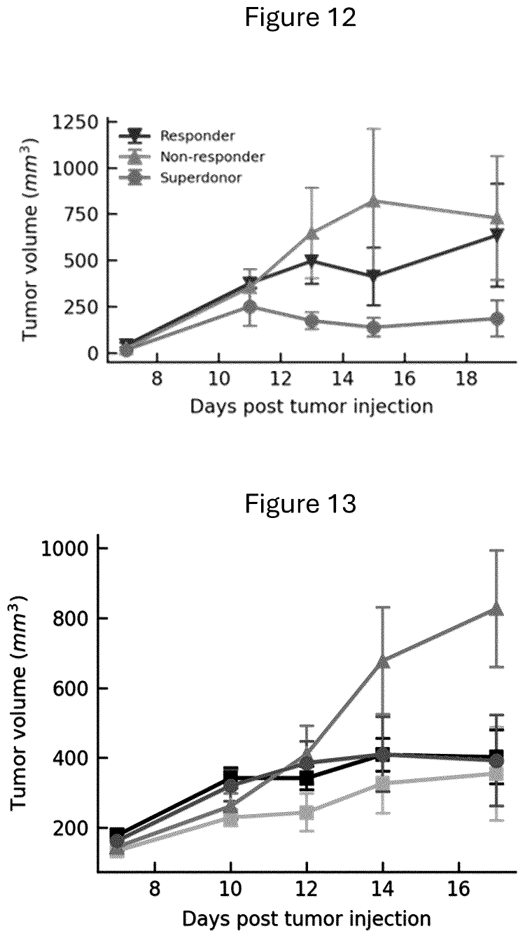

In one study, super donor FMT induced better tumor control with ICI treatment in a melanoma germ-free BP melanoma mouse model as compared to FMT from an ICI non-responder and a benchmark responder. Results are shown in FIG. 12. The super donor's microbiome, administered as a fecal suspension was able to better inhibit tumor growth in combination with ICI treatment compared with either an ICI non-responder or a benchmark responder.

Eight week old female C57BL/6J germ-free mice received a slurry containing 20 mg FMT in a phosphate-buffered saline solution (PBS) with 1000 glycerol (w/v). Mice were orally gavaged QD with 200 μL of the slurry for 3 days every other day. 800,000 BP melanoma cells were injected subcutaneously in the right flank of each mouse 7 days post-FMT. Once tumors reached an average size of 250 mm3, typically day 10-12 after tumor injection, mice were then dosed with 3 doses of 200 g αPD-L1 antibody, administered intraperitoneally every other day. A final tumor measurement was performed at the end of study, 4 days following the last dose of αPD-L1 antibody.

In another study, Consortia 1 and 2, each as set forth in Table 3, treatments were compared to super donor FMT using the same BP melanoma mouse model and ICI treatment as above. Consortia 1, Consortia 2, and super donor fecal material were administered as a suspension (20 mg in 200 μl in PBS with 10% glycerol (w/v)), and tumor growth was compared with mice treated with FMT from an ICI non-responder. The data are shown in FIG. 13 and demonstrate the ability of the microbial consortia treatment to inhibit tumor growth in combination with ICI in similar fashion to the super donor treatment and in greater effect as compared to the benchmark ICI-non-responder treatment.

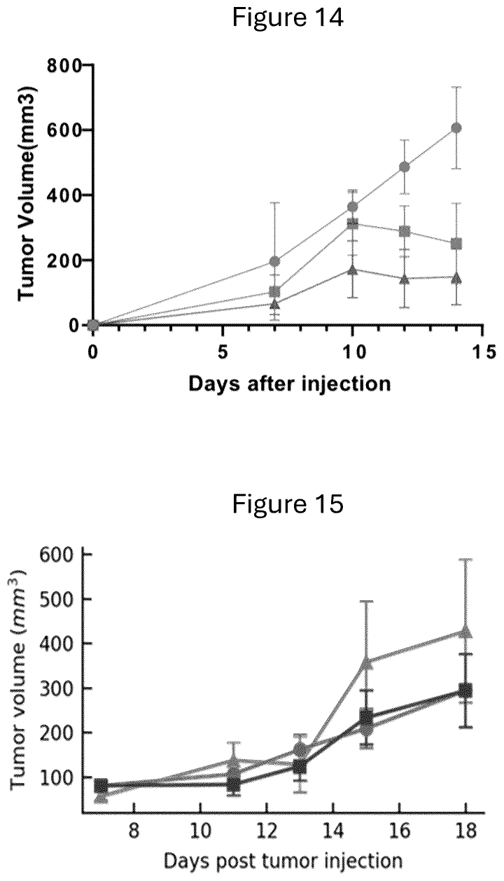

The same methodology with the BP melanoma mouse model was conducted using Consortia 2 and Consortia 5, each as set forth in Table 3, as well as a non-responder FMT control. Results are shown in FIG. 14 and show a similar pattern as in the previous study, with the microbial consortia reducing tumor volume as compared to control.

The microbial consortium of the invention performs in multiple mouse models, including in a specific pathogen free mouse model of colorectal cancer. In this model, 8-week-old female C57BL/6J specific-pathogen-free mice were first treated with antibiotics to deplete their endogenous microbiome. The antibiotic treatment consisted of neomycin (100 mg/kg), vancomycin (50 mg/kg), streptomycin (50 mg/kg), colistin (10 mg/kg), and amphotericin B (1 mg/kg), delivered as a 200 uL oral gavage every 12 hours for 7 days. The following day the mice received a slurry containing 20 mg FMT or Consortia 2, as set forth in Table 3, in PBS with 10% Glycerol (w/v). Mice were orally gavaged QD three times with 200 μL of the slurry every other day. 800,000 MC38 cells were injected subcutaneously in the right flank of each mouse 7 days post-FMT or microbial consortium treatment. Once tumors reached an average size of 250 mm3, typically day 10-12 after tumor injection, mice were then dosed 3 times with 200 μg αPD-L1 antibody, administered intraperitoneally every other day. A final tumor measurement was performed at the end of study, 4 days following the last dose of αPD-L1 antibody.

The results are shown in FIG. 15. These data demonstrate the ability of Consortia 2 to act in a similar fashion to the complex super donor fecal material and inhibit tumor growth in combination with ICI relative to the benchmark non-responder control, in a mouse model that had a pre-existing microbiome. These data also demonstrate the ability of the microbial consortium to impact tumor growth in a model of colorectal cancer.

In another study, 8-week-old female C57BL/6 germ-free mice received a slurry containing 100 mg/mL FMT or microbial consortium in PBS twice on 2 consecutive days. Mice were orally gavaged QD with 200 μL of the slurry during the first administration and 100 μL during the second administration. 800,000 MCA-205 tumor cells were injected subcutaneously in the right flank of each mouse 14 days after the last FMT or microbial consortium treatment. Six days after tumor cell treatment, mice were then dosed with 4 doses of 250 μg αPD-1 antibody, administered intraperitoneally every third day. A final tumor measurement was performed at the end of study, 2 days following the last dose of αPD-1 antibody.

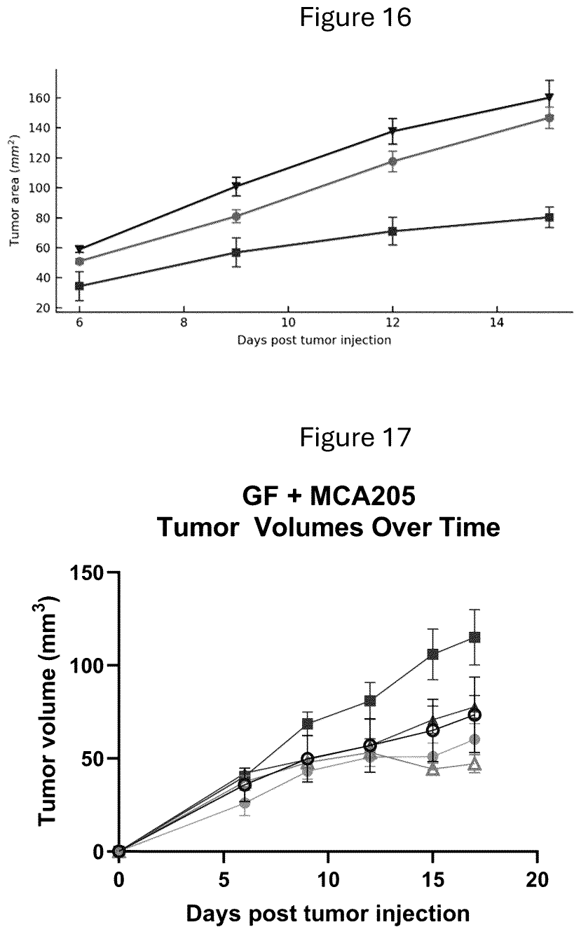

The results are shown in FIG. 16. These data demonstrate the of Consortia 2, as set forth in Table 3, to inhibit tumor growth to an even greater extent than the complex super donor fecal material and the fecal material from the ICI responder. These data also demonstrate the ability of this microbial consortium to impact tumor growth in a fibrosarcoma mouse cancer model.

This methodology using the MCA205 tumor model was also conducted in a study comparing the effects of treatment with Consortia2, 3, 4, and 6, each as set forth in Table 3, on tumor volume, as compared to fecal suspension from a benchmark ICI nonresponder. All microbial consortia reduced tumor volume as compared to the nonresponder control. Results are shown in FIG. 17.

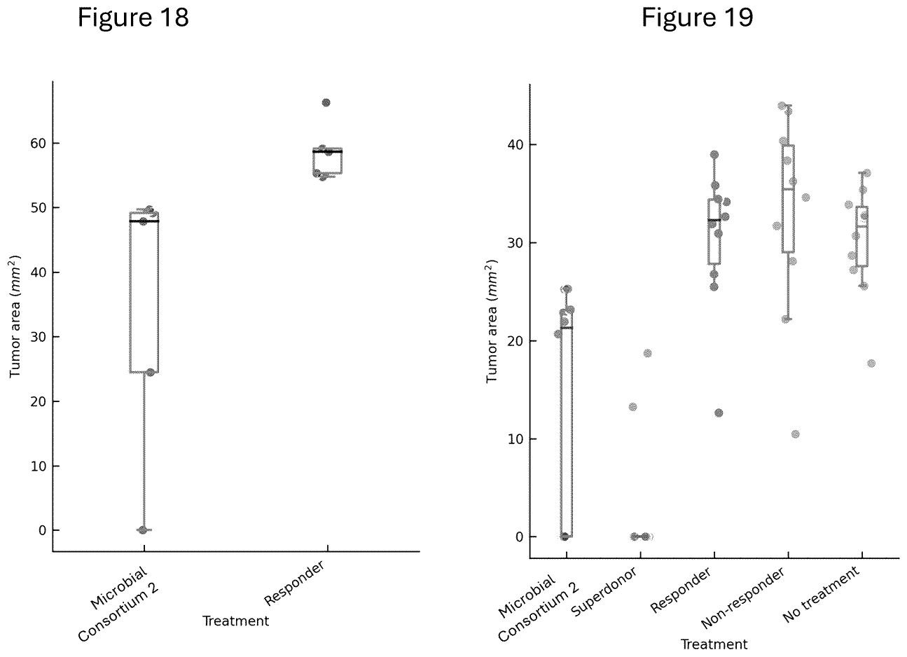

Consortia 2 was shown to reduce tumor prior burden prior to ICI treatment in multiple mouse models. FIG. 18 data depicts the smaller tumor sizes in the Consortia 2 group at day 6 after tumor cell administration, prior to ICI administration. The day-6 timepoint shows each mouse as a separate point on the plot.

In another similar study, a specific-pathogen-free mouse model with the EO771 breast adenocarcinoma cell line was used. In this model, 7-week-old female C57BL/6J specific-pathogen-free mice were first treated with antibiotics to deplete their endogenous microbiome. The following antibiotics were provided to mice in their drinking water for 3 days: ampicillin (1 mg/mL), streptomycin (5 mg/mL), and colistin (1 mg/mL). After a 1-day washout period, the mice received a slurry containing 100 mg/mL FMT or Microbial Consortium 2 in PBS. Mice were orally gavaged QD with 200 μL of the slurry during treatment #1 and 100 μL during treatment #2. Two weeks after the last FMT or microbial consortium treatment, 500,000 EO771 tumor cells were injected subcutaneously in each mouse, and tumor growth was monitored.

Results are shown in FIG. 19 demonstrating tumor volume 10 days post-FMT or microbial consortium treatment. Tumor volume was lower in the Consortis 2-treated group and the super donor treated group as compared to ICI responder, non-responder and no treatment groups.

### Example 6: Microbial Consortia and Engraftment

Mouse engraftment data was used to support bacterial inclusion or exclusion in microbial consortia. Five strains showed poor engraftment and overlap with the non-typical gut commensals described herein.

Human engraftment data was also collected in the MD Anderson Cancer Center clinical study NCT04792322 using sequencing data obtained from metagenomic sequencing of stool samples following FMT from the super donor.

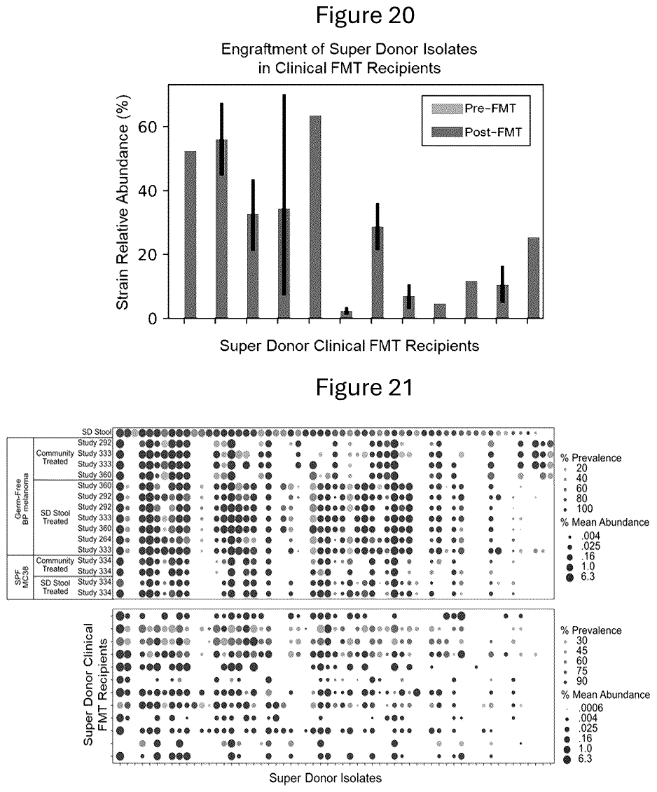

FIG. 20 shows results from the clinical study pre- and post-FMT. Prior to FMT from the super donor, the clinical study patient samples were studied to detect bacteria specifically identified from the super donor isolates. As expected, the values were near zero. Post-FMT analysis, however, shows substantial engraftment of isolates from the super donor in the fecal samples from the clinical trial patients.

The strains identified in the super donor fecal samples made up a significant proportion of each recipient's total microbiome.

Data was also compared on isolate engraftment across both the human clinical study patients and the mouse models. The comparison is shown in the FIG. 21 plot. The upper plot shows engraftment in various mouse models treated with Microbial Consortium 1 and Microbial Consortium 2 as compared to treatment with super donor FMT. The correlate values for super donor stool is aligned at the top of the plot. Each vertical column represents a bacterial isolate. The lower plot shows the engraftment results in human studies in which ICI cancer patients received super donor FMT. Relatively similar engraftment patterns are seen across the human and mouse engraftment data following super donor FMT as well as in mice treated with the microbial consortia.

### Example 7: Increased Tumor Infiltrating Cytotoxic Immune Cells

Tumor transcriptomic data suggest that the microbial consortia of the invention promote the infiltration of cytotoxic CD8 T cells and NK cells and also promote the reduction of pro-tumor M2 macrophage signatures and immune checkpoint expression with which they are associated. In tumors from ICI treated mice who received previous administration of fecal suspension or microbial consortium, the prominent cells in the tumors were T cells, B cells and myeloid cells in non-responder fecal-suspension treated mice, T and NK cells and myeloid cells in superdonor treated mice, and NK cells and CD8 T cells in microbial consortium treated mice. Cytotoxicity profiles were highest in the microbial consortium treated mice. Immune checkpoint expression was lowest in the microbial treated mice. RNA expression profiling also demonstrated an increase in genes mediating an interferon response in non-responder treated mice, and, in the super donor treated and microbial consortium treated mice, an increase in gene expression related to olfactory signaling and antimicrobial response.

### Example 8: Phylogenetic Trees

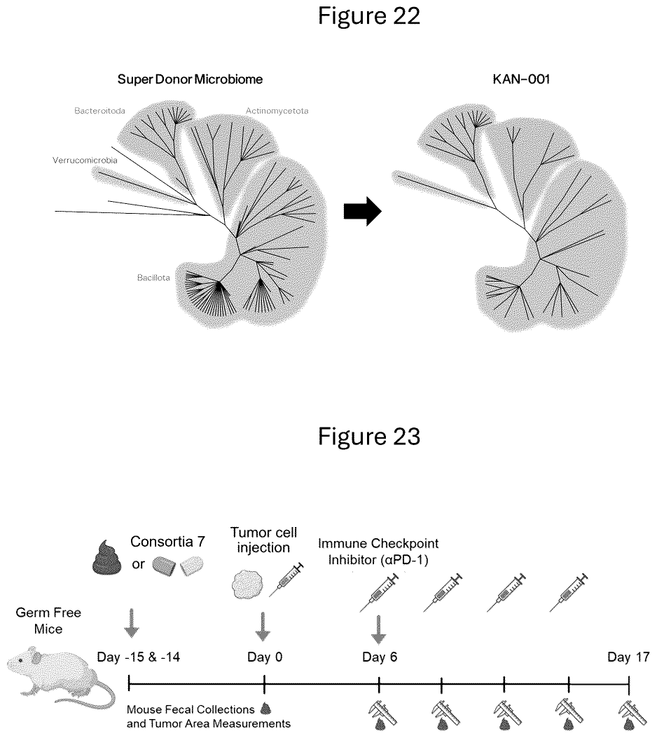

Phylogenetic trees were constructed from all super donor donations to construct the super donor microbiome and for all bacterial species exceeding 0.1% relative abundance (Super Donor Microbiome, see FIG. 22, left hand picture) and for the species isolated, cultured, and banked (Super Donor Isolates, see FIG. 22, right hand picture). The key phyla are shaded in four groups, starting with Bacillota in the lower right and, moving in a counterclockwise direction, Bacteriodota, Actinomycetota, and Verrucomicrobiota. The representation is similar between Super Donor Microbiome and Super Donor Isolates.

### Example 9: Efficacy of Consortia 7

A study was conducted to evaluate the ability of Consortia 7 to induce inhibition of tumor growth in combination with anti-programmed cell death protein 1(PD-1) ICI. Germ-free mice were first engrafted with a candidate consortium and then challenged with MCA205 fibrosarcoma cells before being treated with ICI. As depicted in FIG. 23, six-to-eight-week-old female C57BL/6 GF mice receive a microbial mixture, either as a slurry containing 100 mg/mL of stool in a PBS solution, or Consortia 7, prepared as described in the section below.

Mice were orally gavaged with 200 μL of the consortium or fecal-derived microbial mixture QD on Day −15, and 100 μL on Day −14. 800,000 MCA205 tumor cells were injected s.c. in the right flank of each mouse 14 days post-microbial mixture treatment. Six days after tumor cell injection, mice were dosed with 250 μg anti-PD-1 antibody, administered i.p. every third day. A final tumor measurement was performed at the EOS, 2 days following the last dose of anti-PD-1. Fecal material for metagenomic engraftment analysis was collected from each mouse following microbial engraftment at the time of tumor injection and/or at the EOS.

To prepare Consortia 7 for oral gavage and colonization, two research cell bank (RCB) vials of each strain comprising candidate consortia were anaerobically pooled into a sterile container. The average viable cell count (VCC) of the pooled material was approximately 6×108 cells/mL. After gentle resuspension, the mixtures were aliquoted into 0.9 mL aliquots for cryopreservation at ≤−65° C. On the day of administration, the input candidate consortia were thawed and immediately introduced by gavage as a cell suspension. Each study included a negative control microbiome comprised of fecal material from cancer patients who did not respond to ICI therapy, which served as a benchmark for poor tumor control. The NR stool for the MCA205 model was sourced from a NR non-small cell lung cancer (NSCLC) patient.

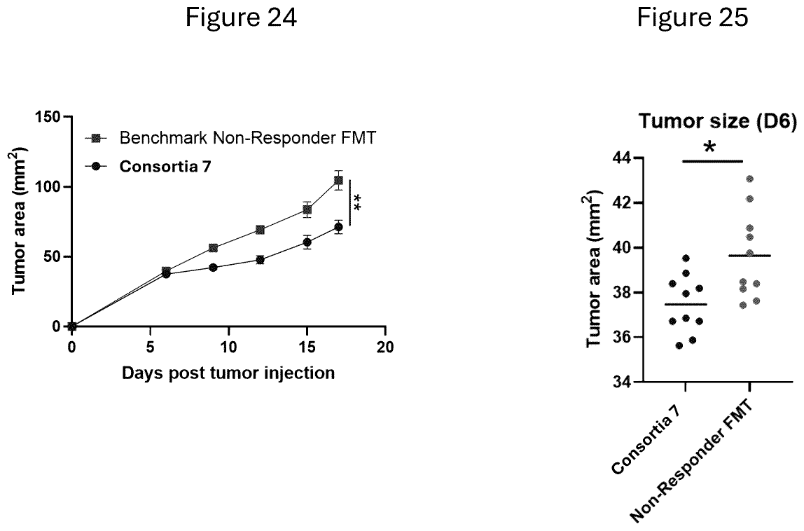

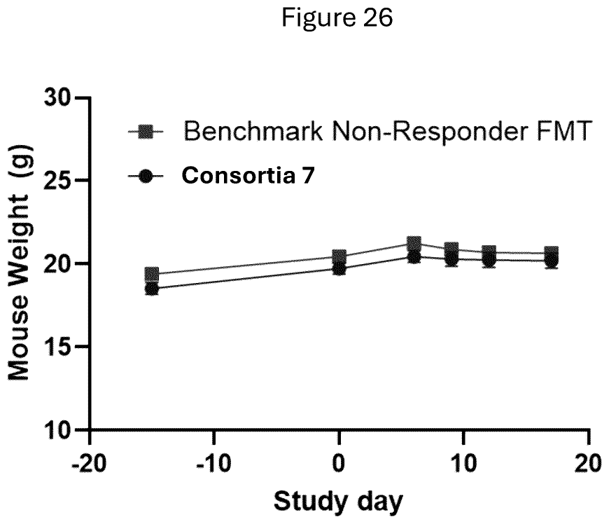

Results of this experiment are shown in FIG. 24-26. FIG. 24 shows a comparison of the ability of Consortia 7 to reduce tumor area compared to the control of a non-responder FMT over time, showing a significant trend of reduced tumor size post dosing. FIG. 25 shows the tumor size comparison of treatment with Consortia 7 compared to a non-responder FMT at day 6. FIG. 26 shows that there was no negative effect on the weight of the mice upon administration of Consortia 7.

### Example 10: Manufacturing of Consortia 7

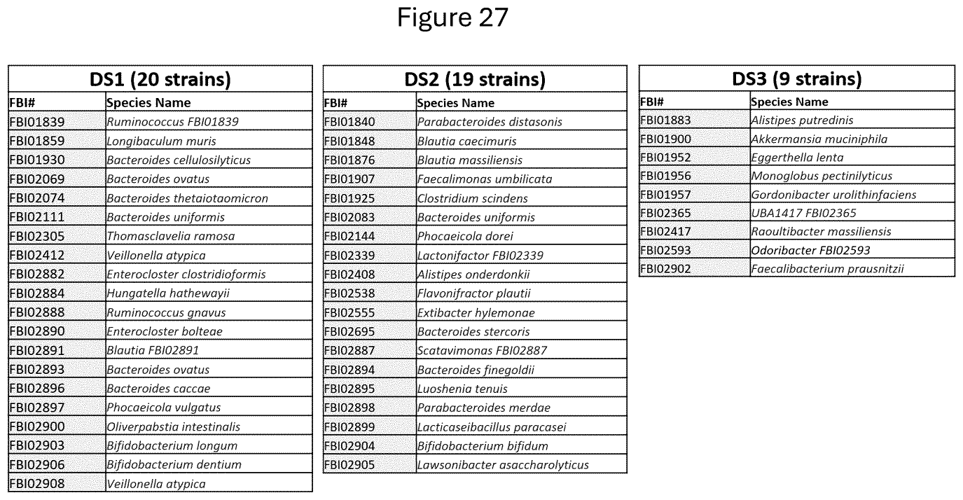

Consortia 7, as set forth in Table 3, was manufactured by growing the strains in 3 different buckets (i.e., drug substances or DSs). FIG. 27 shows the final DSs that were designed for optimal growth.

The optimal growth was determined by the following steps:

1. DS strains were grouped based on growth kinetics and cross feeding/interactions.

2. Modification for relative abundance for Consortia 7 strains by inoculum optimization for each strain at the seed stage (see FIG. 1) and impact on the main fermentation.

3. Modification of biomass yields as well as relative abundance by media optimization and fermentation parameter optimization.

4. Modification for the best cell health at harvest through inoculum densities and functional assay testing.

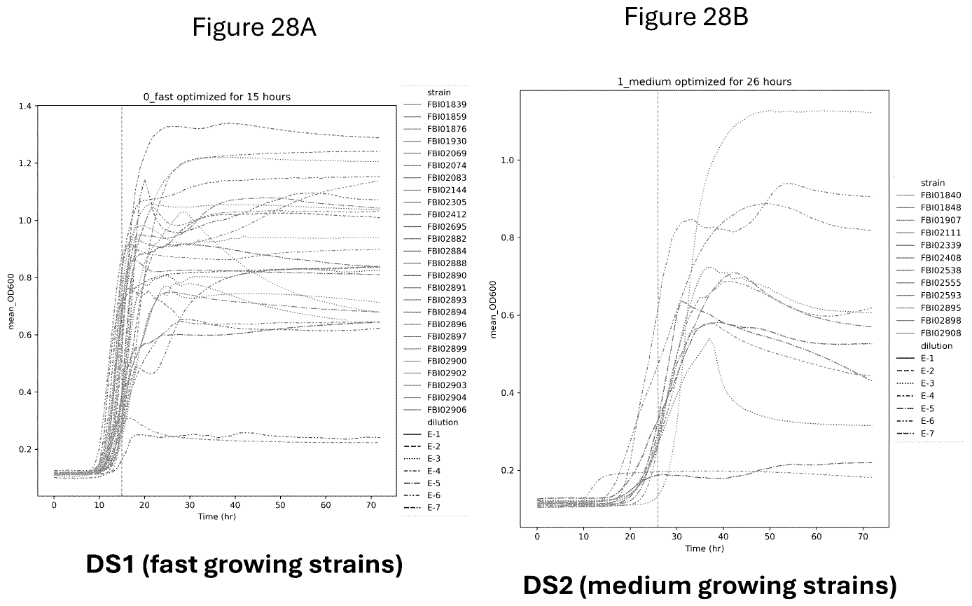

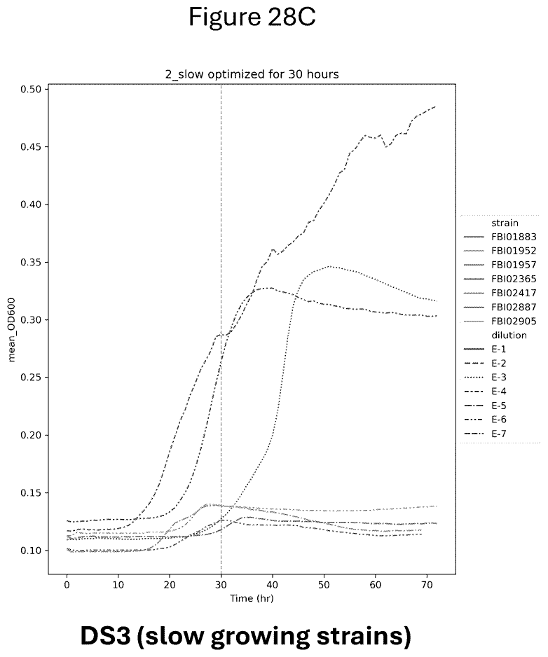

Based on the above described methods, the 3 DSs were designed as follows: DS1 (fast growing strains), DS2 (medium growing strains), and DS3 (slow growing strains). See FIGS. 28A, 28B, and 28C for the growth curves and bucketing of each of the strains of Consortia 7. For this experiment, appropriate dilutions were picked from the growth curves data to obtain similar mid-log growth timing for each strain in the DS bucket.

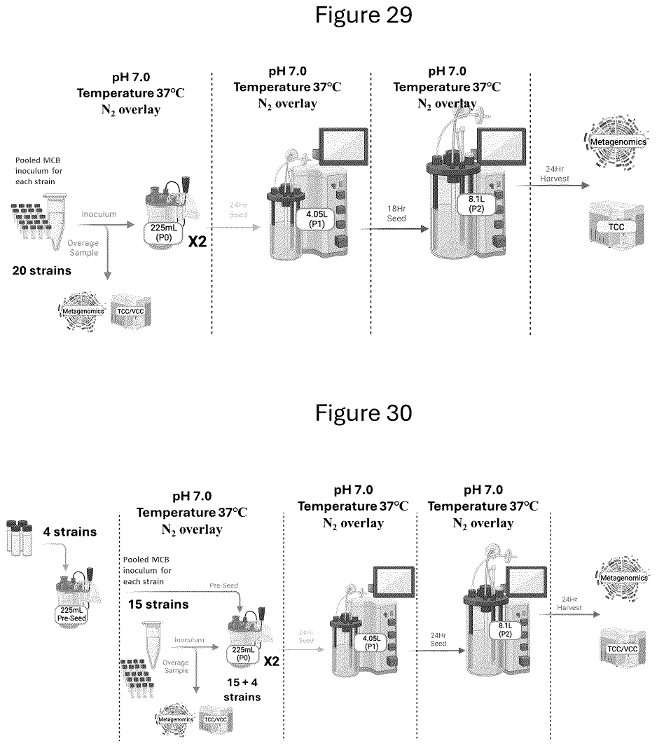

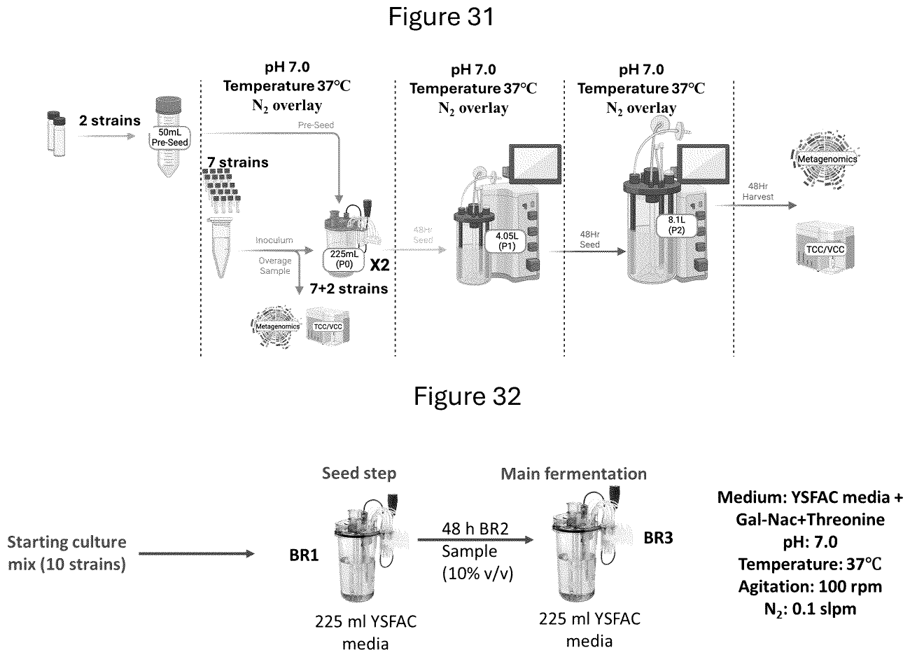

Each of DS1-DS3 was further modified for co-culture fermentation. FIG. 29 depicts the DS1 co-culture fermentation process for the 20 strains included in DS1. FIG. 30 depicts the DS2 co-culture fermentation process for the 19 strains included in DS2. FIG. 31 depicts the DS3 co-culture fermentation process for the 20 strains included in DS3. While YCFAC (Teknova) media was shown to work, a special medium was custom designed. This special medium is termed YSFAC and is a derivative of YCFAC medium with soytone used in place of casitone for improved growth. Furthermore, the medium for DS3 had to be modified to include threonine and N-acetylgalactosamine to promote for growth of the slow-growing strains.

In developing the buckets for the DSs for improved growth, it was observed that Akkermansia muciniphila and Faecalibacterium prusnitzii were particularly slow and finicky strains. They were originally included in both DS1 and DS2; however, neither strain grew in either DS to a robust and detectable density. In order to determine how to grow Faecalibacterium prusnitzii, it was tested in monoculture in a bioreactor to obtain lag times and base draw information. In the bioreactor, Faecalibacterium prusnitzii showed robust growth as a monoculture in YSFAC media. Based on the growth curves, it was determined that Faecalibacterium prusnitzii would be best suited for DS3.

DS3 fermentation was initiated with a starting culture of 10 strains with GalNAc because Akkermansia muciniphila preferentially utilizes GalNAc over glucose as a carbon source and exhibits threonine auxotrophy and N-Acetylgalactosamine is a key component of mucin and serves as the core sugar in mucin O-glycosylation. A depiction of the initial experiment is provided in FIG. 32.

However, the initial attempt of including both Akkermansia muciniphila and Faecalibacterium prusnitzii in DS3 failed (see FIG. 32 for initial process that failed). While Faecalibacterium prusnitzii was detectable using this method, Akkermansia muciniphila was absent or undetectable. Furthermore, Alistipes onderdonkii, which was originally in DS3, was moved to DS2 because despite its initial slow growth curves in monoculture, it outcompeted the other strains in DS3 by a relative 90% abundance and was determined to outcompete Akkermansia muciniphila for GalNac.

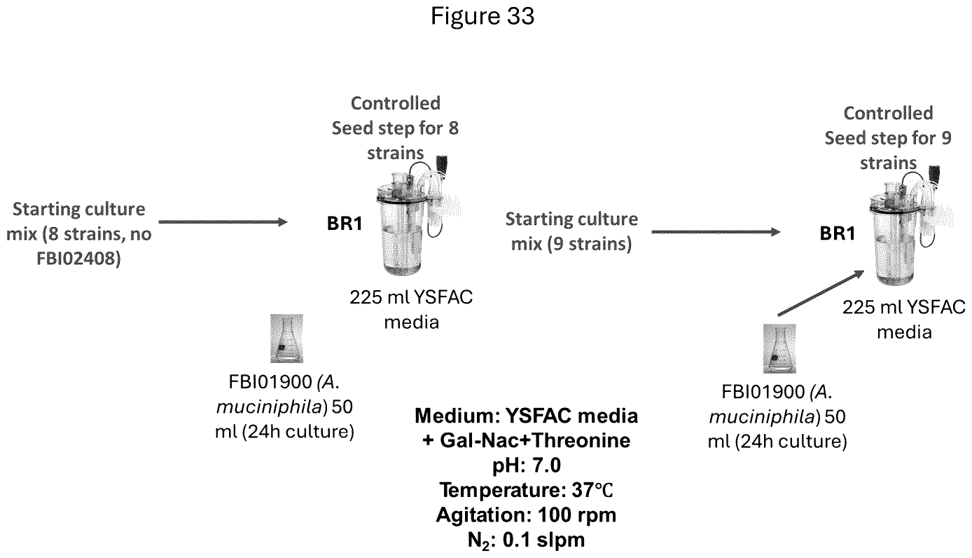

DS3 was further modified by addressing quorum sensing (i.e., the requirement of a certain starting density to grow and thrive) for Akkermansia muciniphila. Accordingly, it was attempted to promote Akkermansia muciniphila growth in DS3 by generating a starting monoculture of Akkermansia muciniphila in a seed flask and adding it to the bioreactor after it reached a certain density. The improved process is depicted in FIG. 33. The improved process was successful and both Akkermansia muciniphila and Faecalibacterium prusnitzii were detectable at harvest in robust abundance.

Furthermore, while the methods of growing Akkermansia muciniphila and Faecalibacterium prusnitzii were developed for DS3, the same method can be used for any monoculture or co-culture method developed for any consortia.

### Example 11: Additional Methods

### Bacteria Isolation

Media was obtained from Anaerobe Systems (Morgan Hill, CA), Teknova (Hollister, CA) and Biochemazone (Leduc, Alberta Canada).

For the conventional method, stool samples from a Sep. 22, 2022 donation from the super donor were serially diluted and mixed with glycerol to a final concentration of 25%, then cryopreserved at ≤−65° C. These serially diluted aliquots were subsequently used for direct plating on agar plates with various microbial cultivation media designed to support anaerobic growth (passage 1), followed by incubation under anaerobic conditions at 37° C. Over the course of several days, the plates were monitored for the emergence of microbial colonies.

Single colonies from these initial plates were picked and transferred to fresh agar plates with appropriate media for further purification (passage 2). After incubation, if single-colony plating resulted in uniform colony morphology, species identification was performed using high-throughput MALDI-time of flight (MALDI-TOF) mass spectrometry. If multiple morphologies appeared, each distinct colony type was selected and plated again on appropriate media for further isolation until uniform morphology was achieved (passage 3 or beyond).

Colonies identified as species of interest were then propagated in liquid culture, stored in cryovials with cryoprotectants to generate purified isolate stocks, and species-confirmed through full-length 16S rRNA gene sequencing or whole-genome sequencing (WGS).

For the MALDI-based liquid culture isolation strategy, fecal material donations from the three separate time points were used. The isolation process began with liquid culture techniques, wherein a single cryovial aliquot of donor stool at the appropriate dilution was thawed and further diluted in media to achieve an estimated one cell per well in 96-well plates. The plates were sealed with plastic film and parafilm, and incubated at 37° C. for 1 to 21 days. Growth was monitored through visual turbidity and optical density measurements. As expected, approximately 30-40% of wells showed no growth, consistent with the target inoculum of one cell per well. Wells with visible growth were harvested, and the cultures were consolidated into a 96-well hit-pick plate.

Once the cultures were on the hit-pick plate, the contents of each well were split into three portions, designated for three specific purposes: (1) cryopreservation of purified isolate stocks, (2) MALDI identification, and (3) high phylogenetic Resolution Florescence (HiPR)-ID identification. For cryopreservation, cultures were mixed with glycerol to a final concentration of 25% and stored at ≤−65° C. for future use. For MALDI identification, cultures were washed with 1×PBS, deposited onto single-use MALDI Biotyper (MBT) biotarget plates embedded in a matrix, and analyzed using a Bruker Microflex LT MALDI-TOF for proteomic characterization and preliminary species-level identification. For each well of interest identified by MALDI, the corresponding cryopreserved portion was reanimated with either growth in liquid media or on solid media and ultimately species-confirmed through full-length 16S rRNA gene sequencing or whole-genome sequencing.

For targeted liquid culture isolation, a December 2023 donation was used. In addition to what is described above for MALDI-based isolation, for HiPR-ID imaging, the cultures were fixed in formaldehyde and stored in 50% ethanol until further analysis. HiPR-ID assays were conducted by depositing cultured samples onto slides, followed by probe hybridization of a panel constructed from taxa uncovered by PacBio 16S sequencing of donor material. A pan-microbial probe (EUB) or DAPI staining was used for general microbial visualization, as most cultures were not expected to exhibit HiPR-ID probe panel signals since the panels targeted specific, often rare, taxa. Samples were visually scanned, and high-interest hits were imaged and analyzed for panel signal and other features to create a list of high-priority wells. Microbial cultures from the corresponding cryovials were streaked onto agar plates; resulting colonies were picked, checked with MALDI, propagated in appropriate liquid culture, and banked in cryovials with cryoprotectant to generate purified isolate stocks. Species were confirmed using full-length 16S rRNA gene sequencing or whole-genome sequencing.

### Sequencing

Short-read Illumina and long-read Nanopore sequencing was collected for each strain using standard extraction and library preparation procedures. Sequences were assembled into high-quality hybrid genome assemblies using Unicycler. Taxonomic assignments were made using GTDB-tk and strains without a species name were given the FBI number as the species name.

### Methods of Culture

Research cell banks (RCBs) were generated from purified isolate stocks. Purified isolate stocks were streaked onto an agar medium which is a modified yeast casitone fatty acids agar with carbohydrates (YCFAC) medium with soytone used in place of casitone (YSFAC). For Akkermansia, YSFAC was additionally supplemented with gal-nac and threonine. Identification was verified with MALDI-TOF spectrometry. Colonies from plates were inoculated into precultures containing appropriate broth media and incubated under anaerobic conditions at 37° C. until sufficient growth was observed. Total cell count (TCC) and percent viability were measured and precultures were used to inoculate main cultures in appropriate broth media. Main cultures were incubated under anaerobic conditions at 37° C. until sufficient growth was observed. TCC and percent viability were measured, and sterile glycerol solution was added to achieve a final glycerol concentration of 25% (v/v) prior to aliquoting approximately 0.9 mL into 2D-barcoded cryovials.

### Growth Curve Studies

Data was collected by growing species in YCFAC media in a 96-well flat bottom plate and measuring OD 600 every hour for 72 hours using an optical plate reader. Growth curve studies were started by thawing one vial of each RCB and creating a serial dilution in 200 μL of YCFAC medium. E-1 designates a 10 fold dilution of the original, E-2 is a 10 fold dilution of E-1 (1/100th of the original solution) and so on for 7 serial dilutions. Each individual curve is the average of three replicates.

Other embodiments are within the scope of the following claims.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1.-93. (canceled)

94. A microbial consortium comprising or consisting of:
(a) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica;
(b) FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908;
(c) Ruminococcus FBI01839, Parabacteroides distasonis, Blautia caecimuris, Longibaculum muris, Streptococcus salivarius, Blautia wexlerae FBI01866, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Enterococcus avium, Streptococcus mutans, Bacteroides uniformis FBI02111, Phocaeicola dorei, Streptococcus parasanguinis, Streptococcus FBI02176, Streptococcus parasanguinis, Enterococcus gallinarum, Streptococcus gallolyticus, Thomasclavelia ramose, Bacteroides thetaiotaomicron, Lactonifactor FBI02339, UBA1417 FBI02365, Alistipes onderdonkii, Veillonella atypica FBI02412, Extibacter hylemonae, Odoribacter FBI02593, and Bacteroides stercoris;
(d) FBI01839, FBI01840, FBI01848, FBI01859, FBI01863, FBI01866, FBI01876, FBI01883, FBI01900, FBI01907, FBI01925, FBI01930, FBI01952, FBI01956, FBI01957, FBI02069, FBI02074, FBI02083, FBI02101, FBI02106, FBI02111, FBI02144, FBI02158, FBI02176, FBI02208, FBI02271, FBI02288, FBI02305, FBI02325, FBI02339, FBI02365, FBI02408, FBI02412, FBI02555, FBI02593, and FBI02695;
(e) Ruminococcus FBI01839, Parabacteroides distasonis, Blautia caecimuris, Longibaculum muris, Streptococcus salivarius, Blautia wexlerae FBI01866, Blautia massiliensis, Alistipes putredinis, Akkermansia muciniphila, Faecalimonas umbilicate, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Enterococcus avium, Streptococcus mutans, Bacteroides uniformis FBI02111, Phocaeicola dorei, Streptococcus parasanguinis, Streptococcus FBI02176, Streptococcus parasanguinis, Enterococcus gallinarum, Streptococcus gallolyticus, Thomasclavelia ramosa, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, and Bacteroides stercoris; or
(f) FBI01839, FBI01840, FBI01848, FBI01859, FBI01863, FBI01866, FBI01876, FBI01883, FBI01900, FBI01907, FBI01925, FBI01930, FBI01952, FBI01956, FBI01957, FBI02069, FBI02074, FBI02083, FBI02101, FBI02106, FBI02111, FBI02144, FBI02158, FBI02176, FBI02208, FBI02271, FBI02288, FBI02305, FBI02325, FBI02339, FBI02365, FBI02408, FBI02412, FBI02417, FBI02538, FBI02555, FBI02593, and FBI02695;
(g) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Bacteroides uniformis FBI02111, Phocaeicola dorei, Thomasclavelia ramosa, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, and Bacteroides stercoris;
(h) FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, and FBI02695;
(i) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Bacteroides uniformis FBI02111, Phocaeicola dorei, Thomasclavelia ramosa, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, Bacteroides stercoris, and Faecalibacterium prausnitzii;
(j) FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, and FBI02902;
(k) Ruminococcus FBI01839, Parabacteroides distasonis, Blautia caecimuris, Longibaculum muris, Blautia massiliensis, Alistipes, putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis FBI02083, Bacteroides uniformis FBI02111, Phocaeicola dorei, Thomasclavelia ramosa, Bacteroides thetaiotaomicron, Lactonifactor FBI02339, UBA1417 FBI02365, Alistipes onderdonkii, Veillonella atypica FBI02412, Raoultibacter massiliensis, Flavonifractor plautii, Extibacter hylemonae, Odoribacter FBI02593, Bacteroides stercoris, Blautia wexlerae FBI01861, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, Veillonella atypica FBI02908, and Enterococcus avium;
(l) FBI01839, FBI01840, FBI01848, FBI01859, FBI01861, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, FBI02908, and FBI02101;
(m) Ruminococcus FBI01839, Parabacteroides distasonis, Blautia caecimuris, Longibaculum muris, Blautia wexlerae, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica; or
(n) FBI01839, FBI01840, FBI01848, FBI01859, FBI01861, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908.

95. A microbial consortium comprising or consisting of the species or the bacterial strains listed in (a) Consortia 5.1 in Table 3, (b) Consortia 6.1 in Table 3, (c) Consortia 6.2 in Table 3, (d) Consortia 1 in Table 3, (e) Consortia 2 in Table 3, (f) Consortia 3, in Table 3, (g) Consortia 4 in Table 3, (h) Consortia 5 in Table 3, (i) Consortia 6 in Table 3, (j) Consortia 7 in Table 3.

96. A microbial consortium comprising:
(a) at least 39 bacterial strains selected from the group consisting of FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908;
(b) at least 29 bacterial strains selected from the group consisting of FBI01839, FBI01840, FBI01848, FBI01859, FBI01863, FBI01866, FBI01876, FBI01883, FBI01900, FBI01907, FBI01925, FBI01930, FBI01952, FBI01956, FBI01957, FBI02069, FBI02074, FBI02083, FBI02101, FBI02106, FBI02111, FBI02144, FBI02158, FBI02176, FBI02208, FBI02271, FBI02288, FBI02305, FBI02325, FBI02339, FBI02365, FBI02408, FBI02412, FBI02555, FBI02593, and FBI02695;
(c) at least 30 bacterial strains selected from the group consisting of FBI01839, FBI01840, FBI01848, FBI01859, FBI01863, FBI01866, FBI01876, FBI01883, FBI01900, FBI01907, FBI01925, FBI01930, FBI01952, FBI01956, FBI01957, FBI02069, FBI02074, FBI02083, FBI02101, FBI02106, FBI02111, FBI02144, FBI02158, FBI02176, FBI02208, FBI02271, FBI02288, FBI02305, FBI02325, FBI02339, FBI02365, FBI02408, FBI02412, FBI02417, FBI02538, FBI02555, FBI02593, and FBI02695;
(d) at least 23 bacterial strains selected from the group consisting of FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, and FBI02695;
(e) at least 24 bacterial strains selected from the group consisting of FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, and FBI02902;
(f) at least 40 bacterial strains selected from the group consisting of FBI01839, FBI01840, FBI01848, FBI01859, FBI01861; FBI01876, FBI01883, FBI01900, FBI01907, FBI01925, FBI01930, FBI01952, FBI01956, FBI01957, FBI02069, FBI02074, FBI02083, FBI02101, FBI02111, FBI02144, FBI02305, FBI02325, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908;
(g) at least 40 bacterial strains selected from the group consisting of FBI01839, FBI01840, FBI01848, FBI01859, FBI01861, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02325, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908;
(h) at least 39 bacterial strains selected from the bacterial strains listed in Consortia 6.2 in Table 3;
(i) at least 40 bacterial strains selected from the bacterial strains listed in Consortia 6.1 in Table 3; or
(j) at least 40 bacterial strains selected from the bacterial strains listed in Consortia 5.1 in Table 3.

97. A microbial consortium comprising or consisting of bacterial strains or species having identical functions or substantially identical functions to the strains of the microbial consortium of claim 96.

98. The microbial consortium of claim 96, wherein each strain of the consortium comprises a 16s RNA that is at least 80% identical to a nucleotide sequence selected from SEQ ID NOs: 1-720.

99. The microbial consortium of claim 96, wherein each strain comprises a 16s RNA selected from SEQ ID NOs: 1-720.

100. The microbial consortium of claim 95, wherein each strain of Consortia 1, 2, 3, 4, 5, 6, or 7 as set forth in Table 3 comprises a 16s RNA selected from SEQ ID NOs: 1-720.

101. A pharmaceutical composition comprising the microbial consortium of claim 96.

102. A food product comprising the microbial consortium of claim 96.

103. The food product of claim 102, further comprising a prebiotic.

104. A method of treating a cancer in a subject in need thereof, slowing tumor growth in a subject in need thereof, or reducing tumor size in a subject in need thereof, the method comprising administering an effective amount of the microbial consortium of claim 96, a pharmaceutical composition thereof, or a food product thereof.

105. The method of claim 104, wherein the subject has received an immunotherapy.

106. The method of claim 105, wherein the subject has a tumor or cancer relapse.

107. The method of claim 104, wherein the subject is a non-responder to immunotherapy.

108. The method of claim 107, wherein the immunotherapy comprises an immune checkpoint inhibitor (ICI) comprising an anti-PD1 antibody, an anti-PD-L1 antibody, an anti-CTLA-4 antibody, an anti-BTLA antibody, an anti-TIM3 antibody, an anti-LAG-3 antibody, or a combination thereof.

109. The method of claim 108, wherein (i) the anti-PD1 antibody comprises pembrolizumab, nivolumab, cemiplimab, dostarlimab, retifanlimab-dlwr, tislelizumab, or combinations thereof, (ii) the anti-CTLA4 antibody comprises ipilimumab or tremelimumab, or a combination thereof, and (iii) the anti-PD-L1 antibody comprises atezolizumab, avelumab, durvalumab, or a combination thereof.

110. The method of claim 104, wherein the tumor or cancer is selected from prostate cancer, liver cancer, renal cancer, lung cancer, breast cancer, colorectal cancer, pancreatic cancer, brain cancer, hepatocellular carcinoma, lymphoma, leukemia, gastric cancer, cervical cancer, ovarian cancer, thyroid cancer, melanoma, head and neck cancer, skin cancer, and soft tissue sarcoma.

111. The method of claim 104, wherein the tumor or cancer is metastatic or malignant selected from melanoma, mismatch repair-deficient/microsatellite instability-high (dMMR/MSI-H) colorectal cancer, non-small cell lung cancer, renal cell carcinoma, head and neck squamous cell carcinoma, urothelial carcinoma, cervical cancer, and triple-negative breast cancer.

112. A kit comprising the microbial consortium of claim 96, a pharmaceutical composition thereof, or a food product thereof.

113. A method of manufacturing a microbial consortium comprising a co-culture method described herein, wherein the microbial consortium comprises or consists of:
(a) Ruminococcus FBI01839, Parabacteroides distasonis, Blautia caecimuris, Longibaculum muris, Blautia massiliensis, Alistipes putredinis, Akkermansia muciniphila, Faecalimonas umbilicata, Clostridium scindens, Bacteroides cellulosilyticus, Eggerthella lenta, Monoglobus pectinilyticus, Gordonibacter urolithinfaciens, Bacteroides ovatus, Bacteroides thetaiotaomicron, Bacteroides uniformis, Phocaeicola dorei, Thomasclavelia ramosa, Lactonifactor FBI02339, UBA1417 FBI02365, Alistipes onderdonkii, Veillonella atypica, Raoultibacter massiliensis, Flavonifractor plautii, Extibacter hylemonae, Odoribacter FBI02593, Bacteroides stercoris, Enterocloster clostridioformis, Hungatella hathewayii, Scatavimonas FBI02887, Ruminococcus gnavus, Enterocloster bolteae, Blautia FBI02891, Bacteroides finegoldii, Luoshenia tenuis, Bacteroides caccae, Phocaeicola vulgatus, Parabacteroides merdae, Lacticaseibacillus paracasei, Oliverpabstia intestinalis, Faecalibacterium prausnitzii, Bifidobacterium longum, Bifidobacterium bifidum, Lawsonibacter asaccharolyticus, Bifidobacterium dentium, and Veillonella atypica; or
(b) FBI01839, FBI01840, FBI01848, FBI01859, FBI01876, FBI01883, FBI01900, FBI01907, FBI01925, FBI01930, FBI01952, FBI01956, FBI01957, FBI02069, FBI02074, FBI02083, FBI02111, FBI02144, FBI02305, FBI02339, FBI02365, FBI02408, FBI02412, FBI02417, FBI02538, FBI02555, FBI02593, FBI02695, FBI02882, FBI02884, FBI02887, FBI02888, FBI02890, FBI02891, FBI02893, FBI02894, FBI02895, FBI02896, FBI02897, FBI02898, FBI02899, FBI02900, FBI02902, FBI02903, FBI02904, FBI02905, FBI02906, and FBI02908.

114. A method of growing Akkermansia muciniphila in a co-culture described herein.
